(19) United States
(12) Patent Application Publication
(10) Pub. No.: US 2017/0189402 A1
(43) Pub. Date: Jul. 6, 2017

(54) SOLID DOSAGE FORMS

(71) Applicant: AbbVie Inc., North Chicago, IL (US)

(72) Inventors: Todd MCDERMOTT, Libertyville, IL (US); Benjamin M. COLLMAN, Wilmette, IL (US); Weili E. WANG, Buffalo Grove, IL (US); Peter T. MAYER, Libertyville, IL (US); Albert SANTIAGO, Beach Park, IL (US); Leana O'KEEFFE, County Cork (IE); John B. MORRIS, Grayslake, IL (US)

(21) Appl. No.: 15/246,279

(22) Filed: Aug. 24, 2016

Related U.S. Application Data
(63) Continuation of application No. 13/851,808, filed on Mar. 27, 2013, now abandoned.
(60) Provisional application No. 61/646,630, filed on May 14, 2012.

(51) Int. Cl. A61K 31/513; A61K 9/28; A61K 9/20; A61K 9/24; A61K 31/427

(52) U.S. Cl. CPC A61K 31/513; A61K 9/209; A61K 31/427; A61K 9/2027; A61K 9/2013; A61K 9/28

(57) ABSTRACT

The present invention features solid dosage forms comprising two or more different active ingredients. In one embodiment, the present invention features a tablet dosage form comprising a first layer and a second layer, wherein the first layer comprises (1) 200 mg lopinavir, (2) ritonavir, (3) a pharmaceutically acceptable hydrophilic polymer, (4) a pharmaceutically acceptable surfactant, and (5) no more than 0.2% by weight of one or more lubricants, and the second layer comprises another therapeutic agent.

### CROSS-REFERENCES TO RELATED APPLICATIONS

This application claims the benefit of U.S. Provisional Application No. 61/616,711, filed Mar. 28, 2012, U.S. Provisional Application No. 61/645,700, filed May 11, 2012, U.S. Provisional Application No. 61/646,630, filed May 14, 2012, and U.S. patent application Ser. No. 13/851,808, filed Mar. 27, 2013.

### FIELD OF THE INVENTION

The present invention relates to solid dosage forms comprising two or more different active ingredients.

### BACKGROUND

Kaletra® (marketed as Aluvia® in developing countries) is one of the most affordable and successful HIV protease inhibitors in the world. Kaletra/Aluvia is a co-formulation of lopinavir and ritonavir, in which lopinavir is the primary HIV protease inhibitor, and ritonavir functions as a pharmacokinetic booster for lopinavir. Upon administration, ritonavir suppresses the lopinavir metabolism mediated by cytochrome P450 3A4 (CYP3A4), thereby increasing the plasma concentration of lopinavir.

Co-formulating lopinavir and ritonavir in solid dosage forms presented extraordinary challenges. Both ritonavir and lopinavir are poorly soluble. In addition, ritonavir is considered a Biopharmaceutics Classification System (BCS) Class IV compound, and BCS Class IV compounds are among the most difficult to be formulated in solid dosage forms.

Prior to 2005, Kaletra was only available in liquid forms in which lopinavir and ritonavir were dissolved in organic solvents. These formulations are unstable at high temperatures and often require refrigerated storage conditions. In addition, these formulations need to be taken with food in order to provide adequate bioavailability. For patients residing in economically challenged or developing countries, these requirements represent a particularly challenging dilemma.

In 2005, after intensive research and major formulation breakthroughs, a heat-stable Kaletra tablet was developed and approved. The heat-stable Kaletra tablet contains 200 mg of lopinavir and 50 mg of ritonavir, and provides critically important advantages for patients in developing countries, including storage without refrigeration, no dietary restrictions, and lower pill burden.

Lamivudine (3TC) is a potent nucleoside analog reverse transcriptase inhibitor. Lamivudine is administered orally with a bioavailability of over 80%. Lamivudine is commercially available as Epivir® tablet, one form of which contains 150 mg of crystalline lamivudine.

### SUMMARY OF THE INVENTION

Combining another therapeutic agent such as lamivudine with lopinavir and ritonavir in a single solid dosage form presents significant challenges. The release mechanism of the active ingredients from Kaletra tablet is different from many other drugs. Therefore, a simple mixture of Kaletra tablet with another drug is unlikely to achieve bioequivalence with respect to lopinavir and the other drug. On the other hand, attempts to add another drug layer to Kaletra tablet have failed to produce a mechanically stable bilayer tablet with a size suitable for oral administration. The present invention unexpectedly found that by reducing the lubricant content in Kaletra tablet, a mechanically stable bilayer or multiple-layer tablet can be successfully prepared.

Accordingly, in one aspect, the present invention features a tablet dosage form comprising a first layer and a second layer, wherein the first layer comprises (1) from 160 to 200 mg lopinavir (e.g., 170 mg lopinavir, or 180 mg lopinavir, or 200 mg lopinavir), (2) ritonavir, (3) a pharmaceutically acceptable hydrophilic polymer, (4) a pharmaceutically acceptable surfactant and (5) no more than 0.9% by weight of one or more lubricants, and the second layer comprises another therapeutic agent (e.g., 75 mg lamivudine).

Preferably, the first layer of the tablet dosage form comprises no more than 0.5% by weight of any lubricants. More preferably, the first layer comprises no more than 0.2% by weight of any lubricants. Highly preferably, the first layer comprises no more than 0.1% by weight of any lubricants.

In one embodiment, the weight ratio of the second layer to the first layer is no more than 1:4; for example, the weight ratio of the second layer to the first layer can be no more than 1:5. Preferably, the first layer comprises no more than 0.2% by weight of any lubricants.

In yet another embodiment, the tablet dosage form is no more than 1.7 g, and the first layer is at least 1 g; for instance, the dosage form is no more than 1.6 g, and the first layer is at least 1 g; for another instance, the dosage form is no more than 1.5 g, and the first layer is at least 1 g; and for yet another instance, the dosage form is no more than 1.5 g, and the first layer is at least 1.1 g. Preferably, the first layer comprises no more than 0.2% by weight of any lubricants.

In another embodiment, the first layer includes a solid dispersion comprising ritonavir, lopinavir, said hydrophilic polymer and said surfactant; for instance, the first layer comprises a solid solution, said solution comprising ritonavir, lopinavir, said hydrophilic polymer and said surfactant. Preferably, the first layer comprises no more than 0.2% by weight of any lubricants.

In another embodiment, the first layer comprises a glassy solution, said glassy comprising ritonavir, lopinavir, said hydrophilic polymer and said surfactant. In one example, said hydrophilic polymer is copovidone, and said surfactant is sorbitan monolaurate. Preferably, the lubricant(s) is sodium stearyl fumarate. Preferably, the weight ratio of lopinavir to ritonavir in the first layer is 4:1. Also preferably, the first layer comprises no more than 0.2% by weight of any lubricants.

In another embodiment, the tablet dosage form is no more than 1.5 g, the first layer is at least 1.1 g, and the weight ratio of the second layer to the first layer is no more than 1:4; for instance, the weight ratio of the second layer to the first layer is no more than 1:5. Preferably, the first layer comprises no more than 0.2% by weight of any lubricants.

In another embodiment, a tablet dosage form of this aspect of the invention comprises 170 mg lopinavir. As used herein, a dosage form of an aspect of the invention includes any tablet dosage form described in the embodiments or examples of the aspect of the invention. In another embodiment, a tablet dosage form of this aspect of the invention comprises 180 mg lopinavir. In another embodiment, a tablet dosage form of this aspect of the invention comprises 200 mg lopinavir.

In yet another embodiment, a tablet dosage form of this aspect of the invention comprises 50 mg ritonavir, 200 mg lopinavir, and 75 mg lamivudine. In another embodiment, a tablet dosage form of this aspect of the invention comprises 45 mg ritonavir, 180 mg lopinavir, and 75 mg lamivudine. In another embodiment, a tablet dosage form of this aspect of the invention comprises 42.5 mg ritonavir, 170 mg lopinavir, and 75 mg lamivudine. In another embodiment, a tablet dosage form of this aspect of the invention comprises 40 mg ritonavir, 160 mg lopinavir, and 75 mg lamivudine.

In another aspect, the present invention features tablet dosage forms suitable for pediatric use. Such a tablet dosage form comprises a first layer and a second layer, wherein the first layer comprises (1) from 80 to 100 mg lopinavir (e.g., 85 mg lopinavir, or 90 mg lopinavir, or 100 mg lopinavir), (2) ritonavir, (3) a pharmaceutically acceptable hydrophilic polymer, (4) a pharmaceutically acceptable surfactant and (5) no more than 0.9% by weight of one or more lubricants, and the second layer comprises another therapeutic agent (e.g., 37.5 mg lamivudine). Preferably, the first layer comprises no more than 0.5% by weight of any lubricants. More preferably, the first layer comprises no more than 0.2% by weight of any lubricants. Highly preferably, the first layer comprises no more than 0.1% by weight of any lubricants.

In one embodiment of this aspect of the invention, the weight ratio of the second layer to the first layer is no more than 1:4; for example, the weight ratio of the second layer to the first layer can be no more than 1:5. Preferably, the first layer comprises no more than 0.2% by weight of any lubricants.

In yet another embodiment of this aspect of the invention, the tablet dosage form is no more than 0.85 g, and the first layer is at least 0.5 g; for instance, the tablet dosage form is no more than 0.8 g, and the first layer is at least 0.5 g; for another instance, the tablet dosage form is no more than 0.75 g, and the first layer is at least 0.5 g; and for yet another instance, the tablet dosage form is no more than 0.75 g, and the first layer is at least 0.55 g. Preferably, the first layer comprises no more than 0.2% by weight of any lubricants.

In another embodiment of this aspect of the invention, the first layer includes a solid dispersion comprising ritonavir, lopinavir, said hydrophilic polymer and said surfactant; for instance, the first layer comprises a solid solution, said solution comprising ritonavir, lopinavir, said hydrophilic polymer and said surfactant. Preferably, the first layer comprises no more than 0.2% by weight of any lubricants.

In another embodiment of this aspect of the invention, the first layer comprises a glassy solution, said glassy comprising ritonavir, lopinavir, said hydrophilic polymer and said surfactant. In one example, said hydrophilic polymer is copovidone, and said surfactant is sorbitan monolaurate. Preferably, the lubricant(s) is sodium stearyl fumarate. Preferably, the weight ratio of lopinavir to ritonavir in the first layer is 4:1. Also preferably, the first layer comprises no more than 0.2% by weight of any lubricants.

In another embodiment of this aspect of the invention, the tablet dosage form is no more than 0.75 g, the first layer is at least 0.55 g, and the weight ratio of the second layer to the first layer is no more than 1:4; for instance, the weight ratio of the second layer to the first layer is no more than 1:5. Preferably, the first layer comprises no more than 0.2% by weight of any lubricants.

In another embodiment, a tablet dosage form of this aspect of the invention comprises 85 mg lopinavir. In another embodiment, a tablet dosage form of this aspect of the invention comprises 90 mg lopinavir. In another embodiment, a tablet dosage form of this aspect of the invention comprises 100 mg lopinavir.

In another embodiment, a tablet dosage form of this aspect of the invention comprises 25 mg ritonavir, 100 mg lopinavir, and 37.5 mg lamivudine. In another embodiment, a tablet dosage form of this aspect of the invention comprises 22.5 mg ritonavir, 90 mg lopinavir, and 37.5 mg lamivudine. In another embodiment, a tablet dosage form of this aspect of the invention comprises 21.25 mg ritonavir, 85 mg lopinavir, and 37.5 mg lamivudine. In another embodiment, a tablet dosage form of this aspect of the invention comprises 20 mg ritonavir, 80 mg lopinavir, and 37.5 mg lamivudine.

A pharmaceutically acceptable salt of lopinavir and/or a pharmaceutically acceptable salt of ritonavir can be used in any tablet dosage form of the invention (including a tablet dosage form suitable for pediatric use), in lieu of lopinavir and ritonavir, respectively.

In yet another aspect, the present invention features processes for making a tablet dosage form of the invention. In one embodiment, the process comprises compressing a first layer and a second layer under a compression force of no more than 30 kN and preferably no more than 25 kN, wherein the first layer comprises (1) from 160 to 200 mg lopinavir (e.g., 170 mg lopinavir, or 180 mg lopinavir, or 200 mg lopinavir), (2) ritonavir, (3) a pharmaceutically acceptable hydrophilic polymer, (4) a pharmaceutically acceptable surfactant, and (5) no more than 0.9% by weight of one or more lubricants, and the second layer comprises another therapeutic agent (e.g., 75 mg lamivudine). Any tablet dosage form described herein can be similarly prepared by compressing the respective first and second layers under a compression force of no more than 30 kN and preferably no more than 25 kN.

The present invention further features methods of treating HIV infection. The methods comprise administering a tablet dosage form of the invention to a HIV patient.

In still another aspect, the present invention features a solid pharmaceutical dosage form comprising (1) lopinavir or a pharmaceutically acceptable salt thereof; (2) ritonavir or a pharmaceutically acceptable salt thereof; and (3) lamivudine or a pharmaceutically acceptable salt thereof. The solid pharmaceutical dosage form preferably is a bilayer tablet comprising at least a first layer and a second layer, wherein the first layer comprises (1) ritonavir or pharmaceutically acceptable salt thereof and (2) lopinavir or pharmaceutically acceptable salt thereof, and the second layer comprises lamivudine or pharmaceutically acceptable salt thereof.

In one embodiment of this aspect of the invention, the first layer comprises at least 75% by weight of the bilayer tablet. Preferably, the first layer comprises less than 1.6% by weight of flow regulator, relative to total weight of the first layer.

In one example of this embodiment, the second layer comprises from 21.4% to 50% by weight of lamivudine, based on the weight of the second layer. For instance, the second layer comprises (1) from 0% to 75% by weight of microcrystalline cellulose, based on total weight of the second layer; (2) from 0% to 50% by weight of lactose, based on total weight of the second layer; (3) from 1.0% to 5.0% by weight of sodium starch glycolate, based on total weight of the second layer; (4) from 0.1% to 2.0% by weight of sodium stearyl fumarate, based on total weight of the second layer; (5) from 0.2% to 3.0% by weight of colloidal silicon dioxide, based on total weight of the second layer; and (6) from 0% to 75% by weight of hydroxypropyl cellulose, based on total weight of the second layer.

In another example of this embodiment, the second layer is essentially free of lactose. In still another example of this embodiment, the second layer comprises (1) from 60.1% to 60.3% by weight of microcrystalline cellulose; (2) 3.0% by weight of sodium starch glycolate; (3) 0.8% by weight of sodium stearyl fumarate; and (4) from 0.2% to 0.4% by weight of colloidal silicon dioxide.

In another embodiment of this aspect of the invention, the first layer comprises (1) from 65% to 75% by weight of at least one pharmaceutically acceptable water-soluble polymer, (2) from 4% to 10% by weight of at least one pharmaceutically acceptable surfactant, (3) from 0.01% to 1% by weight of at least one lubricant, and (4) from 0.01% to 3% by weight of at least one glidant, wherein all % by weight are based on the total weight of the first layer. Preferably, the first layer comprises less than 1% by weight of a lubricant (e.g., less than 0.5% by weight, or preferably less than 0.2% by weight), relative to total weight of the first layer.

In another embodiment of this aspect of the invention, the first layer comprises (1) 65% to 75% by weight of N-vinyl pyrrolidone/vinyl acetate copolymer 60:40, (2) from 3% to 10% by weight of sorbitan monolaurate, (3) from 0.05% to less than 1% by weight of sodium stearyl fumarate, and (4) from 1% to 1.2% by weight of colloidal silicon dioxide, all % by weight being based on the total weight of the first layer.

In yet another embodiment, a solid dosage form of this aspect of the invention comprises 50 mg ritonavir, 200 mg lopinavir, and 75 mg lamivudine. In another embodiment, a solid dosage form of this aspect of the invention comprises 45 mg ritonavir, 180 mg lopinavir, and 75 mg lamivudine. In another embodiment, a solid dosage form of this aspect of the invention comprises 42.5 mg ritonavir, 170 mg lopinavir, and 75 mg lamivudine. In another embodiment, a solid dosage form of this aspect of the invention comprises 40 mg ritonavir, 160 mg lopinavir, and 75 mg lamivudine.

In another embodiment, a solid dosage form of this aspect of the invention comprises 25 mg ritonavir, 100 mg lopinavir, and 37.5 mg lamivudine. In another embodiment, a solid dosage form of this aspect of the invention comprises 22.5 mg ritonavir, 90 mg lopinavir, and 37.5 mg lamivudine. In another embodiment, a solid dosage form of this aspect of the invention comprises 21.25 mg ritonavir, 85 mg lopinavir, and 37.5 mg lamivudine. In another embodiment, a solid dosage form of this aspect of the invention comprises 20 mg ritonavir, 80 mg lopinavir, and 37.5 mg lamivudine.

In still another embodiment of this aspect of the invention, the solid dosage form provides (1) an in-vitro release profile of lamivudine or salt thereof, wherein at least 95% of lamivudine or salt thereof is released from the dosage form within ten minutes, (2) an in-vitro release profile of lopinavir or salt thereof, wherein at least 80% of lopinavir or salt thereof is released from the dosage form within 90 minutes, and 100% is released from the dosage form within 120 minutes, and (3) an in-vitro release profile of ritonavir or salt thereof, wherein at least 80% of ritonavir or salt thereof is released from the dosage form within 90 minutes, and 100% is released from the dosage form within 120 minutes. The in vitro release profiles can be determined using a USP type 2 dissolution apparatus, at 75 rpm in 900 ml of 0.06M POE10LE for 120 minutes at 37° C.

In still another embodiment of this aspect of the invention, the solid pharmaceutical dosage form comprise (1) ritonavir or a pharmaceutically acceptable salt thereof; (2) lopinavir or a pharmaceutically acceptable salt thereof; (3) from 55% to 75% by weight of N-vinyl pyrrolidone/vinyl acetate copolymer 60:40, based on total weight of the dosage form; (4) from 4% to 10% by weight of sorbitan monolaurate, based on total weight of the dosage form; (5) from 0.01% to 1% by weight of sodium stearyl fumarate, based on total weight of the dosage form; and (6) from 0.01% to 1.6% by weight of colloidal silicon dioxide, based on total weight of the dosage form.

The present invention also features methods of treating HIV infection, comprising administering a solid pharmaceutical dosage form of this aspect of the invention to a mammal in need of such treatment.

The present invention further features methods for preparing a solid pharmaceutical dosage form. In one aspect, the methods comprise (1) preparing a homogeneous melt comprising (i) ritonavir or pharmaceutically acceptable salt thereof, (ii) lopinavir or pharmaceutically acceptable salt thereof, (iii) a pharmaceutically acceptable water-soluble polymer(s), (iv) a pharmaceutically acceptable surfactant(s) and (v) a flow regulator(s); (2) extruding the melt to obtain an extrudate, with optional milling of the extrudate; (3) mixing the extrudate with a flow regulator and a lubricant to form a first pharmaceutical preparation; (4) adding the first pharmaceutical preparation to a bilayer tablet press; (5) adding a second pharmaceutical preparation to the bilayer tablet press, wherein the second pharmaceutical preparation comprises lamivudine or pharmaceutically acceptable salt thereof; (6) compressing the first pharmaceutical preparation and the second pharmaceutical preparation together to form a bilayer tablet.

In one embodiment of this aspect of the invention, the amount of lubricant is less than 1%, based on the total weight of the dosage form. In another embodiment, the amount of flow regulator mixed with the extrudate is 0.6% by weight or less, based on total weight of the extrudate. In yet another embodiment, the methods comprise initially compressing the first pharmaceutical preparation before compressing the first and second pharmaceutical preparations together, wherein the initial compressing step is at a lower pressure than the compression of the first pharmaceutical preparation and second pharmaceutical preparation together.

In still another aspect, the present invention features solid pharmaceutical dosage forms comprising (a) lopinavir or a pharmaceutically acceptable salt thereof; (b) ritonavir or a pharmaceutically acceptable salt thereof; and (c) an additional ant-HIV agent or a pharmaceutically acceptable salt thereof. Preferably, the dosage form is a bilayer tablet comprising at least a first layer and a second layer, wherein the first layer comprises the ritonavir or pharmaceutically acceptable salt thereof and the lopinavir or pharmaceutically acceptable salt thereof, and the second layer comprises the additional anti-HIV agent or pharmaceutically acceptable salt thereof.

In one embodiment of this aspect of the invention, the first layer comprises at least 75% by weight of the bilayer tablet. In another embodiment, the additional anti-HIV agent is selected from the group consisting of nucleoside reverse transcriptase inhibitors, non-nucleoside reverse transcriptase inhibitors, entry or fusion inhibitors, and mixtures thereof. In still another embodiment, the additional anti-HIV agent is one or more agents selected from the group consisting of atazanavir, darunavir, amprenavir, fosamprenavir, indinavir, nelfinavir, saquinavir, tipranavir, lamivudine, abacavir, stavudine, didanosine, zidovudine, emtricitabine, tenofovir, delavirdine, efavirenz, rilpivirine, etravirine, nevirapine, enfuvirtide, maraviroc, raltegravir, and pharmaceutically acceptable salts thereof.

In a further embodiment of this aspect of the invention, the first layer comprises (1) from 55% to 75% by weight of at least one pharmaceutically acceptable water-soluble polymer, based on total weight of the first layer; (2) from 4% to 10% by weight of at least one pharmaceutically acceptable surfactant, based on total weight of the first layer; (3) from 0.01% to 1% by weight of at least one lubricant, based on total weight of the first layer; and (4) from 0.01% to 3% by weight of at least one glidant, based on total weight of the first layer. In one example of this embodiment, the first layer comprises less than 0.1% by weight of a lubricant, relative to total weight of the first layer. In another example of this embodiment, the solid dosage form comprises less than 0.5% by weight of a lubricant, relative to total weight of the dosage form.

In another embodiment of this aspect of the invention, the first layer comprises less than 1.6% by weight of flow regulator, relative to total weight of the first layer.

In still another embodiment of this aspect of the invention, the second layer comprises from 21.4% to 50% by weight of the additional anti-HIV agent, based on the weight of the second layer. For instance, the second layer comprises (1) from 0% to 75% by weight of microcrystalline cellulose, based on total weight of the second layer; from 0% to 50% by weight of lactose, based on total weight of the second layer; from 1.0% to 5.0% by weight of sodium starch glycolate, based on total weight of the second layer; from 0.1% to 2.0% by weight of sodium stearyl fumarate, based on total weight of the second layer; from 0.2% to 3.0% by weight of colloidal silicon dioxide, based on total weight of the second layer; and from 0% to 75% by weight of hydroxypropyl cellulose, based on total weight of the second layer.

In another embodiment of this aspect of the invention, the second layer has a weight of 300 mg or less. In still another embodiment, the second layer is essentially free of lactose. In another embodiment, the second layer comprises from 60.1% to 60.3% by weight of microcrystalline cellulose; 3.0% by weight of sodium starch glycolate; 0.8% by weight of sodium stearyl fumarate; and from 0.2% to 0.4% by weight of colloidal silicon dioxide.

The present invention also features methods of treating HIV infection, comprising administering a solid pharmaceutical dosage form of this aspect of the invention to a mammal in need of such treatment.

In another aspect, the present invention features methods of preparing a solid pharmaceutical dosage form comprising lopinavir, ritonavir, and an additional anti-HIV agent, or a pharmaceutically acceptable salt of one or more of the foregoing. The methods comprise (1) preparing a homogeneous melt comprising (i) ritonavir or pharmaceutically acceptable salt thereof, (ii) lopinavir or pharmaceutically acceptable salt thereof, (iii) a pharmaceutically acceptable water-soluble polymer(s), (iv) a pharmaceutically acceptable surfactant(s) and (v) a flow regulator(s); (2) extruding the melt to obtain an extrudate, with optional milling of the extrudate; (3) mixing the extrudate with a flow regulator and a lubricant to form a first pharmaceutical preparation; (4) adding the first pharmaceutical preparation to a bilayer tablet press; (5) adding a second pharmaceutical preparation to the bilayer tablet press, wherein the second pharmaceutical preparation comprises an additional anti-HIV agent or pharmaceutically acceptable salt thereof; and (6) compressing the first pharmaceutical preparation and the second pharmaceutical preparation together to form a bilayer tablet. In one embodiment, the amount of lubricant mixed with the extrudate is less than 1%, based on the total weight of the dosage form. In another embodiment, the amount of flow regulator mixed with the extrudate is 0.6% by weight or less, based on total weight of the extrudate. In still another embodiment, the methods comprise initially compressing the first pharmaceutical preparation before compressing the first and second pharmaceutical preparations together, wherein the initial compressing is at a lower pressure than the compression of the first pharmaceutical preparation and second pharmaceutical preparation together.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

Lopinavir/ritonavir and lamivudine have not been successfully combined in a solid, fixed-dose combination formulation that is bioequivalent or substantially similar to a combination of Kaletra and Epivir tablets. Epivir tablet is an immediate release formulation and can be prepared by blending crystalline lamivudine with other excipients. In contrast, Kaletra tablet is an erosion-based formulation and is prepared by dispersing lopinavir and ritonavir in a polymer-based matrix to form an amorphous solid dispersion. Therefore, when these two formulations are mixed together, the release profile of each active ingredient is expected to be significantly altered, thereby producing a composition that is unlikely bioequivalent or substantially similar to a combination of Kaletra and Epivir tablets.

Compressing Epivir and Kaletra tablets into a single, bilayer tablet was also found to be exceedingly difficult. Kaletra tablet has a large tablet size due to the use of a large amount of polymers in order to confer adequate bioavailability to lopinavir and ritonavir. For instance, the commercial Kaletra tablet containing 200 mg lopinavir has a typical size of over 1,200 mg. A bilayer tablet often requires the two layers of active ingredients to be of similar sizes in order to provide manufacturability and/or physical stability. This would require an increase in the lamivudine layer to about 1,200 mg, making the eventual bilayer tablet about 2,400 mg, which is too large to be orally administered.

On the other hand, reducing the size of the lamivudine layer would lead to less manufacturability and/or physical stability of the bilayer tablet. For instance, many attempts to compress a 200-300 mg lamivudine/excipient mixture (containing 75 mg lamivudine) against a 1,200 mg lopinavir/ritonavir tablet core (containing 200 mg lopinavir) failed to produce a stable bilayer tablet. In many cases, the lamivudine layer is mechanically unstable and readily forms capping, lamination or other defects.

It was unexpectedly found that when the lubricant content in the lopinavir/ritonavir tablet core is reduced, a mechanically stable bilayer tablet can be successfully produced, leading to a stable tablet dosage form that contains both a lopinavir/ritonavir layer and a lamivudine layer (or another drug layer) and is suitable for oral administration.

Accordingly, in one aspect, the present invention features a tablet dosage form comprising a first layer and a second layer. The first layer comprises (i) from 160 to 200 mg lopinavir (e.g., 170 mg lopinavir, or 180 mg lopinavir, or 200 mg lopinavir), (ii) ritonavir, (iii) a pharmaceutically acceptable hydrophilic polymer, (iv) a pharmaceutically acceptable surfactant, and (v) no more than 0.9% by weight of one or more lubricants; and the second layer comprises another therapeutic agent (e.g., 75 mg lamivudine). Preferably, the first layer contains no more than 0.5% by weight of any lubricants. More preferably, the first layer contains no more than 0.2% by weight of any lubricants. Highly preferably, the first layer contains no more than 0.1% by weight of any lubricants. As used herein, the term “% by weight”, when used in describing the content of a component in a tablet layer, refers to the weight percentage of the component in that layer. A commercial Kaletra tablet contains about 1% by weight of sodium stearyl fumarate as lubricant. See Example 3 of U.S. Pat. No. 8,025,899, which describes a post-extrusion process comprising blending 12.3 parts by weight of sodium stearyl fumarate (as lubricant) with about 1,200 mg milled extrudate; and a similar process is used for the manufacturing of Kaletra tablets.

The weight ratio of lopinavir to ritonavir in the first layer can be, for example and without limitation, 4:1. For instance, the first layer can comprise 50 mg ritonavir and 200 mg lopinavir, or 45 mg ritonavir and 180 mg lopinavir, or 42.5 mg ritonavir and 170 mg lopinavir, or 40 mg ritonavir and 160 mg lopinavir.

The weight ratio of the second layer to the first layer can be, for example and without limitation, no more than 1:4. Preferably, the weight ratio of the second layer to the first layer is no more than 1:5; for example, the weight ratio of the second layer to the first layer can be 1:5, 1:6 or less. Also preferably, the first layer comprises at least 60% weight of the tablet dosage form. More preferably, the first layer comprises at least 70% weight of the tablet dosage form. Highly preferably, the first layer comprises at least 80% weight of the tablet dosage form.

A tablet dosage form of the invention can be, for example and without limitation, from 1.3 g to 1.7 g, and the first layer can be, for example and without limitation, from 0.9 g to 1.2 g. For instance, the tablet dosage form can be from 1.3 g to 1.5 g, and the first layer is from 1.0 g to 1.2 g. For instance, the tablet dosage form can be from 1.4 g to 1.6 g, and the first layer is from 1.1 g to 1.3 g. In one example, the tablet dosage form is no more than 1.7 g, and the first layer is at least 1 g. In another example, the tablet dosage form can be no more than 1.6 g, and the first layer is at least 1 g. In another yet example, the tablet dosage form can be no more than 1.5 g, and the first layer is at least 1 g. In yet another example, the tablet dosage form is no more than 1.7 g, and the first layer is at least 1.1 g. In yet another example, the tablet dosage form is no more than 1.6 g, and the first layer is at least 1.1 g. In yet another example, the tablet dosage form is no more than 1.5 g, and the first layer is at least 1.1 g. In yet another example, the tablet dosage form is no more than 1.7 g, and the first layer is at least 1.2 g. In yet another example, the tablet dosage form is no more than 1.6 g, and the first layer is at least 1.2 g. In yet another example, the tablet dosage form is no more than 1.5 g, and the first layer is at least 1.2 g. In yet another example, the tablet dosage form is no more than 1.7 g, and the first layer is from 1.0 g to 1.3 g. In another example, the tablet dosage form is no more than 1.7 g, and the first layer is at from 1.0 g to 1.2 g. In yet another example, the tablet dosage form is no more than 1.7 g, and the first layer is from 1.0 g to 1.1 g. In yet another example, the tablet dosage form is no more than 1.7 g, and the first layer is from 1.1 g to 1.3 g. In yet another example, the tablet dosage form is no more than 1.7 g, and the first layer is from 1.1 g to 1.2 g. In yet another example, the tablet dosage form is no more than 1.6 g, and the first layer is from 1.0 g to 1.3 g. In another example, the tablet dosage form is no more than 1.6 g, and the first layer is at from 1.0 g to 1.2 g. In yet another example, the tablet dosage form is no more than 1.6 g, and the first layer is from 1.0 g to 1.1 g. In yet another example, the tablet dosage form is no more than 1.6 g, and the first layer is from 1.1 g to 1.3 g. In yet another example, the tablet dosage form is no more than 1.6 g, and the first layer is from 1.1 g to 1.2 g. In yet another example, the tablet dosage form is no more than 1.5 g, and the first layer is from 1.0 g to 1.3 g. In another example, the tablet dosage form is no more than 1.5 g, and the first layer is at from 1.0 g to 1.2 g. In yet another example, the tablet dosage form is no more than 1.5 g, and the first layer is from 1.0 g to 1.1 g. In yet another example, the tablet dosage form is no more than 1.5 g, and the first layer is from 1.1 g to 1.3 g. In yet another example, the tablet dosage form is no more than 1.5 g, and the first layer is from 1.1 g to 1.2 g.

The first layer preferably includes a solid dispersion, where the solid dispersion comprises ritonavir, lopinavir, the hydrophilic polymer, and the surfactant. More preferably, the first layer comprises a solid solution, where the solid solution comprises ritonavir, lopinavir, the hydrophilic polymer, and the surfactant. Highly preferably, the first layer comprises a glassy solution, where the glassy solution comprises ritonavir, lopinavir, the hydrophilic polymer, and the surfactant.

Lopinavir and ritonavir can also be formulated in different solid dispersions and then mixed and included in the first layer. In one example, the first layer comprises a first and second solid dispersions (i.e., a lopinavir solid dispersion and a ritonavir solid dispersion, respectively), wherein the first solid dispersion comprises lopinavir and a first pharmaceutically acceptable hydrophilic polymer, and the second solid dispersion comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid dispersions (i.e., a lopinavir solid dispersion and a ritonavir solid dispersion, respectively), wherein the first solid dispersion comprises lopinavir, a first pharmaceutically acceptable hydrophilic polymer, and a first pharmaceutically acceptable surfactant, and the second solid dispersion comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In yet another example, the first layer comprises a first and second solid solutions (i.e., a lopinavir solid solution and a ritonavir solid solution, respectively), wherein the first solid solution comprises lopinavir and a first pharmaceutically acceptable hydrophilic polymer, and the second solid solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid solutions (i.e., a lopinavir solid solution and a ritonavir solid solution, respectively), wherein the first solid solution comprises lopinavir, a first pharmaceutically acceptable hydrophilic polymer, and a first pharmaceutically acceptable surfactant, and the second solid solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In still another example, the first layer comprises a first and second glassy solutions (i.e., a lopinavir glassy solution and a ritonavir glassy solution, respectively), wherein the first glassy solution comprises lopinavir and a first pharmaceutically acceptable hydrophilic polymer, and the second glassy solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second glassy solutions (i.e., a lopinavir glassy solution and a ritonavir glassy solution, respectively), wherein the first glassy solution comprises lopinavir, a first pharmaceutically acceptable hydrophilic polymer, and a first pharmaceutically acceptable surfactant, and the second glassy solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

A solid dispersion employed in the present invention preferably comprises or consists of a single-phase (defined in thermodynamics) in which lopinavir and/or ritonavir, together with the pharmaceutically acceptable hydrophilic polymer and/or the pharmaceutically acceptable surfactant, are molecularly dispersed. In such cases, thermal analysis of the solid dispersion using differential scanning calorimetry (DSC) typically shows only one single glass transition temperature (T_{g}), and the solid dispersion does not contain any detectable crystalline lopinavir or ritonavir as measured by X-ray powder diffraction spectroscopy.

In one embodiment, a tablet dosage form of the invention comprises a first layer and a second layer, wherein the first layer comprises 200 mg lopinavir and 50 mg ritonavir and constitutes at least 70% by weight of the total dosage form, and the second layer comprises 75 mg lamivudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 200 mg lopinavir and 50 mg ritonavir and constitutes at least 70% by weight of the total dosage form, and the second layer comprises 75 mg lamivudine and 150 mg zidovudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In yet another embodiment, a tablet dosage form of the invention comprises a first layer and a second layer, wherein the first layer comprises 200 mg lopinavir and 50 mg ritonavir and constitutes at least 70% by weight of the total dosage form, and the second layer comprises 200 mg raltegravir potassium. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 180 mg lopinavir and 45 mg ritonavir and constitutes at least 70% by weight of the total dosage form, and the second layer comprises 75 mg lamivudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 180 mg lopinavir and 45 mg ritonavir and constitutes at least 70% by weight of the total dosage form, and the second layer comprises 75 mg lamivudine and 150 mg zidovudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In yet another embodiment, a tablet dosage form of the invention comprises a first layer and a second layer, wherein the first layer comprises 180 mg lopinavir and 45 mg ritonavir and constitutes at least 70% by weight of the total dosage form, and the second layer comprises 200 mg raltegravir potassium. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 170 mg lopinavir and 42.5 mg ritonavir and constitutes at least 70% by weight of the total dosage form, and the second layer comprises 75 mg lamivudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 170 mg lopinavir and 42.5 mg ritonavir and constitutes at least 70% by weight of the total dosage form, and the second layer comprises 75 mg lamivudine and 150 mg zidovudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In yet another embodiment, a tablet dosage form of the invention comprises a first layer and a second layer, wherein the first layer comprises 170 mg lopinavir and 42.5 mg ritonavir and constitutes at least 70% by weight of the total dosage form, and the second layer comprises 200 mg raltegravir potassium. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 160 mg lopinavir and 40 mg ritonavir and constitutes at least 70% by weight of the total dosage form, and the second layer comprises 75 mg lamivudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In another embodiment, a tablet dosage form of the invention comprises a first layer and a second layer, wherein the first layer comprises 160 mg lopinavir and 40 mg ritonavir and constitutes at least 70% by weight of the total dosage form, and the second layer comprises 75 mg lamivudine and 150 mg zidovudine. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

In yet another embodiment, a tablet dosage form of the invention comprises a first layer and a second layer, wherein the first layer comprises 160 mg lopinavir and 40 mg ritonavir and constitutes at least 70% by weight of the total dosage form, and the second layer comprises 200 mg raltegravir potassium. The first layer comprises (i) at least 50% by weight a pharmaceutically acceptable hydrophilic polymer, (ii) a pharmaceutically acceptable surfactant, and (iii) less than 0.2% by weight of one or more lubricants. Preferably, the first layer contains 0.1% by weight of one or more lubricants. The total weight of the tablet is no more than 1700 mg. Preferably, the tablet is no more than 1650 mg. More preferably, the tablet is no more than 1600 mg. Highly preferably, the tablet is no more than 1580 mg.

A pharmaceutically acceptable salt of lopinavir or a pharmaceutically acceptable salt of ritonavir can be used, instead of lopinavir or ritonavir, respectively, in the first layer.

The other therapeutic agent in the second layer can be, for example and without limitation, an anti-HIV agent, an anti-HCV agent, or another anti-viral agent. Non-limiting examples of the other therapeutic agent include anti-HIV agents, such as lamivudine, zidovudine, didanosine, abacavir, efavirenz, emtricitabine, tenofovir, stavudine, delavirdine, rilpivirine, etravirine, nevirapine, enfuvirtide, maraviroc, raltegravir, dolutegravir, lersivirine, atazanavir, darunavir, amprenavir, fosamprenavir, indinavir, nelfinavir, saquinavir, tipranavir, or a pharmaceutically acceptable salt thereof, or a combination of two or more of the above agents or their respective salts (e.g., a combination of lamivudine and zidovudine, a combination of abacavir, lamivudine and zidovudine, a combination of efavirenz, emtricitabine and tenofovir, a combination of emtricitabine and tenofovir, or a combination of emtricitabine, rilpivirine and tenofovi). Other non-limiting examples of the other therapeutic agent in the second layer include anti-HCV agents, such as NM-811 (Novartis), SCY-635 (Scynexis), ITX-4520 (iTherx), ITX-5061 (iTherx), ANA-773 (Anadys), ABT-072 (Abbott), ABT-333 (Abbott), ANA-598 (Anadys), setrobuvir, BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), ACH-2928 (Achillion), AZD2836 (Astra-Zeneca), AZD7295 (Astra-Zeneca), BMS-790052 (BMS), BMS-824393 (BMS), EDP-239 (Enanta), GS-5885 (Gilead), PPI-1301 (Presidio), PPI-461 (Presidio), GS-6620 (Gilead), IDX-102 (Idenix), IDX-184 (Idenix), INX-189 (Inhibitex), MK-0608 (Merck), PSI-7977 (Pharmasset), PSI-938 (Pharmasset), RG7128 (Roche), TMC64912 (Medivir), GSK625433 (GlaxoSmithKline), BCX-4678 (BioCryst), ABT-450 (Abbott/Enanta), ACH-1095 (Achillion), ACH-1625 (Achillion), ACH-2684 (Achillion), AVL-181 (Avila), AVL-192 (Avila), BI-201335 (Boehringer Ingelheim), BMS-650032 (BMS), boceprevir, danoprevir, GS-9132 (Gilead), GS-9256 (Gilead), GS-9451 (Gilead), IDX-136 (Idenix), IDX-316 (Idenix), IDX-320 (Idenix), MK-5172 (Merck), narlaprevir, PHX-1766 (Phenomix), telaprevir, TMC-435 (Tibotec), vaniprevir, VBY708 (Virobay), VX-500 (Vertex), VX-813 (Vertex), VX-985 (Vertex), CTS-1027 (Conatus), GS-9620 (Gilead), PF-4878691 (Pfizer), RO5303253 (Roche), ALS-2200, ALS-2158, GSK62336805, or a pharmaceutically acceptable salt thereof, or a combination of two or more of the above agents or their respective salts.

The amount of the other therapeutic agent in the second layer can range, for example and without limitation, from 10 mg to 300 mg, such as from 50 mg to 200 mg.

Preferably, the other therapeutic agent in the second layer is a HIV reverse transcriptase inhibitors (e.g., lamivudine or zidovudine), a HIV integrase (e.g., raltegravir or dolutegravir), a HIV entry inhibitor (e.g., enfuvirtide or maraviroc), or a combination thereof (e.g., a combination of lamivudine and zidovudine, a combination of abacavir, lamivudine and zidovudine, a combination of efavirenz, emtricitabine and tenofovir, a combination of emtricitabine and tenofovir, or a combination of emtricitabine, rilpivirine and tenofovir). More preferably, the other therapeutic agent in the second layer comprises lamivudine.

In one embodiment, the second layer in a tablet dosage form of the invention (e.g., any tablet dosage form described herein) comprises 75 mg lamivudine. Preferably, with respect to lopinavir and lamivudine, the tablet dosage form is bioequivalent to a combination of a Kaletra tablet (with 200 mg lopinavir and 50 mg ritonavir per tablet) and one half of an Epivir tablet (with 150 mg lamivudine per tablet). As used herein, a first dosage form is bioequivalent to a second dosage form (or a combination of dosage forms) with respect to an active ingredient if the 90% confidence intervals around the AUC_{∞} point estimate of the active ingredient in the first dosage form, relative to the second dosage form (or the combination of dosage forms), is within the range of from 80% to 125% when tested in a single dose study. A first dosage form is also considered bioequivalent to a second dosage form (or a combination of dosage forms) with respect to an active ingredient if the 90% confidence intervals around the AUCₜ point estimate of the active ingredient in the first dosage form, relative to the second dosage form (or the combination of dosage forms), is within the range of from 80% to 125% when tested in a single or multiple dose study. In a single dose study, t typically is from 24 to 36 hours, such as 24 or 36 hours. In a multiple dose study, t is one complete dosing interval. AUC_{∞} or AUCₜ point estimates can be tested in either humans or dogs (e.g., beagles); preferably AUC_{∞} or AUCₜ point estimates are determined in humans. Moreover, with respect to an active ingredient, a tablet dosage form of invention is considered bioequivalent to a combination of a Kaletra tablet (200 mg lopinavir and 50 mg ritonavir per tablet) and one half of an Epivir tablet (150 mg lamivudine per tablet) if two such tablets of the invention are shown to be bioequivalent to a combination of two Kaletra tablets (200 mg lopinavir and 50 mg ritonavir per tablet) and one Epivir tablet (150 mg lamivudine per tablet).

The present invention unexpected found that by reducing the amount of lubricant in the first layer (the lopinavir/ritonavir tablet core) of a tablet dosage form of the invention, the bioavailability of lopinavir and ritonavir can be improved. As a result, the amount of lopinavir and ritonavir in the first layer can be reduced (e.g., reduced from 200 mg lopinavir and 50 mg ritonavir, to 180 mg lopinavir and 45 mg ritonavir, or 170 mg lopinavir and 42.5 mg ritonavir, or 160 mg lopinavir and 40 mg ritonavir), while the tablet dosage maintains bioequivalence to Kaletra tablet (200 mg lopinavir and 50 mg ritonavir per tablet) with respect to lopinavir. Preferably, the first layer of the tablet comprises no more than 0.2% by weight of any lubricants; more preferably, the first layer comprises 0.1% by weight of lubricant(s).

In another embodiment, the second layer in a tablet dosage form of the invention (e.g., any tablet dosage form described herein) comprises 75 mg lamivudine and 150 mg zidovudine. Preferably, with respect to lopinavir, lamivudine and zidovudine, the tablet dosage form is bioequivalent to a combination of a Kaletra tablet (with 200 mg lopinavir and 50 mg ritonavir per tablet) and one half of a Combivir tablet (with 150 mg lamivudine and 300 mg zidovudine per tablet).

In yet another embodiment, the second layer in a tablet dosage form of the invention (e.g., any tablet dosage form described herein) comprises raltegravir or a pharmaceutically acceptable salt thereof (e.g., raltegravir potassium). Preferably, the second layer comprises 200 mg raltegravir potassium, and the tablet dosage form is bioequivalent to a combination of a Kaletra tablet (with 200 mg lopinavir and 50 mg ritonavir per tablet) and one half of an Isentress® tablet (with 400 mg raltegravir potassium per tablet) with respect to lopinavir and raltegravir potassium.

A pharmaceutically acceptable hydrophilic polymers employed in the first layer of a tablet dosage form of the invention preferably have a T_{g} of at least 50° C. More preferably, the hydrophilic polymer has a T_{g} of at least 60° C., such as at least 80° C., at least 100° C., or from 80° C. to 180° C., or from 100° C. to 150° C. Methods for determining T_{g} values of organic polymers are described in INTRODUCTION TO PHYSICAL POLYMER SCIENCE (2nd Edition by L. H. Sperling, published by John Wiley & Sons, Inc., 1992). The T_{g} value can be calculated as the weighted sum of the T_{g} values for homopolymers derived from each of the individual monomers, i.e., the polymer T_{g}=ΣWᵢ·Xᵢ where Wᵢ is the weight percent of monomer i in the organic polymer, and Xᵢ is the T_{g} value for the homopolymer derived from monomer i. T_{g} values for the homopolymers may be taken from POLYMER HANDBOOK (2nd Edition by J. Brandrup and E. H. Immergut, Editors, published by John Wiley & Sons, Inc., 1975). Hydrophilic polymers with a T_{g} as described above may allow for the preparation of solid dispersions that are mechanically stable and, within ordinary temperature ranges, sufficiently temperature stable so that the solid dispersions may be used as dosage forms without further processing or be compacted to tablets with only a small amount of tabletting aids. Hydrophilic polymers having a T_{g} of below 50° C. may also be included.

A pharmaceutically acceptable hydrophilic polymer employed in the first layer of a tablet dosage form of the invention preferably is water-soluble. The first layer may also include a poorly water-soluble or water-insoluble polymer, such as a cross-linked polymer. A hydrophilic polymer employed in the first layer preferably has an apparent viscosity, when dissolved at 20° C. in an aqueous solution at 2% (w/v), of 1 to 5000 mPa·s., and more preferably of 1 to 700 mPa·s, and most preferably of 5 to 100 mPa·s.

Pharmaceutically acceptable hydrophilic polymers that are suitable for use in the first layer include, but are not limited to, homopolymers or copolymers of N-vinyl lactams, such as homopolymers or copolymers of N-vinyl pyrrolidone (e.g., polyvinylpyrrolidone (PVP), or copolymers of N-vinyl pyrrolidone and vinyl acetate or vinyl propionate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); high molecular polyalkylene oxides, such as polyethylene oxide, polypropylene oxide, and copolymers of ethylene oxide and propylene oxide; polyacrylates or polymethacrylates, such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), and poly(hydroxyalkyl methacrylates); polyacrylamides; vinyl acetate polymers, such as copolymers of vinyl acetate and crotonic acid, and partially hydrolyzed polyvinyl acetate (also referred to as partially saponified “polyvinyl alcohol”); polyvinyl alcohol; oligo- or polysaccharides, such as carrageenans, galactomannans, and xanthan gum; polyhydroxyalkylacrylates; polyhydroxyalkyl-methacrylates; copolymers of methyl methacrylate and acrylic acid; polyethylene glycols (PEGs); or any mixture thereof.

Non-limiting examples of suitable hydrophilic polymers include polyvinylpyrrolidone (PVP) K17, PVP K25, PVP K30, PVP K90, hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, HPMC P 55, Ethocel 4, Ethocel 7, Ethocel 10, Ethocel 14, Ethocel 20, copovidone (vinylpyrrolidone-vinyl acetate copolymer 60/40), polyvinyl acetate, methacrylate/methacrylic acid copolymer (Eudragit®) L100-55, Eudragit L100, Eudragit S100, polyethylene glycol (PEG) 400, PEG 600, PEG 1450, PEG 3350, PEG 4000, PEG 6000, PEG 8000, poloxamer 124, poloxamer 188, poloxamer 237, poloxamer 338, and poloxamer 407.

Of these, homopolymers or copolymers of N-vinyl pyrrolidone, such as copolymers of N-vinyl pyrrolidone and vinyl acetate, are preferred. A non-limiting example of a preferred polymer is a copolymer of 60% by weight of N-vinyl pyrrolidone and 40% by weight of vinyl acetate. Other preferred polymers include, without limitation, hydroxypropyl methylcellulose (HPMC, also known as hypromellose in USP), such as hydroxypropyl methylcellulose grade E5 (HPMC-E5); and hydroxypropyl methylcellulose acetate succinate (HPMC-AS).

Preferably, the first layer in a tablet dosage form of the invention comprises at least 50% by weight of a pharmaceutical acceptable hydrophilic polymer or polymers. More preferably, the first layer comprises at least 60% by weight of a pharmaceutically acceptable hydrophilic polymer or polymers. Highly preferably, the first layer comprises at least 70% weight of a pharmaceutically acceptable hydrophilic polymer or polymers. In one embodiment, the first layer comprises from 60 to 80% weight of a pharmaceutically acceptable hydrophilic polymer or polymers. In another embodiment, the first layer comprises from 65 to 75% weight of a pharmaceutically acceptable hydrophilic polymer or polymers.

In each aspect, example and embodiment described herein, sugar alcohols or other binders may be used in addition to, or in lieu of, hydrophilic polymers in the first layer.

In each aspect, embodiment or example of the invention, a pharmaceutically acceptable surfactant employed in the first layer of a tablet dosage form of the invention preferably is a non-ionic surfactant. Also preferably, a pharmaceutically acceptable surfactant employed in the first layer has an HLB value of from 4 to 10. More preferably, a pharmaceutically acceptable surfactant employed in the first layer has an HLB value of from 7 to 9. The HLB system (Fiedler, H. B., ENCYLOPEDIA OF EXCIPIENTS, 5^{th} ed., Aulendorf: ECV-Editio-Cantor-Verlag (2002)) attributes numeric values to surfactants, with lipophilic substances receiving lower HLB values and hydrophilic substances receiving higher HLB values. A surfactant having an HLB value other than 4-10, such as a surfactant having an HLB value of above 10, may also be used. As used herein, a surfactant encompasses a mixture or combination of two or more different surfactants.

Pharmaceutically acceptable surfactants that are suitable for use in the first layer include, but are not limited to, polyoxyethylene alkyl ether, polyoxyethylene alkylaryl ether, polyethylene glycol fatty acid ester, alkylene glycol fatty acid mono ester, sucrose fatty acid ester, and sorbitan fatty acid mono ester. Non-limiting examples of suitable surfactants include polyoxyethylene (3) lauryl ether, polyoxyethylene (5) cetyl ether, polyoxyethylene (2) stearyl ether, polyoxyethylene (5) stearyl ether, polyoxyethylene (2) nonylphenyl ether, polyoxyethylene (3) nonylphenyl ether, polyoxyethylene (4) nonylphenyl ether, polyoxyethylene (3) octylphenyl ether, PEG-200 monolaurate, PEG-200 dilaurate, PEG-300 dilaurate, PEG-400 dilaurate, PEG-300 distearate, PEG-300 dioleate, propylene glycol monolaurate (e.g., Lauroglycol®), sucrose monostearate, sucrose distearate, sucrose monolaurate, sucrose dilaurate, sorbitan mono laurate (e.g., Span® 20), sorbitan monooleate, sorbitan monopalnitate (e.g., Span® 40), sorbitan stearate, or mixtures of one or more thereof.

The sorbitan mono fatty acid esters are preferred, with sorbitan mono laurate and sorbitan mono palmitate being particularly preferred.

Other pharmaceutically acceptable surfactants that may be used in the first layer include, but are not limited to, polyoxyethylene castor oil derivates, e.g. polyoxyethyleneglycerol triricinoleate or polyoxyl 35 castor oil (Cremophor® EL; BASF Corp.) or polyoxyethyleneglycerol oxystearate such as polyethyleneglycol 40 hydrogenated castor oil (Cremophor® RH 40) or polyethyleneglycol 60 hydrogenated castor oil (Cremophor® RH 60); or block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, Poloxamer® 407 (BASF Wyandotte Corp.); or a mono fatty acid ester of polyoxyethylene (20) sorbitan, e.g. polyoxyethylene (20) sorbitan mono oleate (Tween® 80), polyoxyethylene (20) sorbitan monostearate (Tween® 60), polyoxyethylene (20) sorbitan mono palmitate (Tween® 40), polyoxyethylene (20) sorbitan monolaurate (Tween® 20).

In one embodiment, the first layer comprises two or more surfactants, wherein the surfactant(s) having an HLB value of from 4 to 10 accounts for at least 50% by weight, preferably at least 60% by weight, of the total amount of surfactants used in the first layer.

The first layer preferably comprises at least 1% by weight of a pharmaceutically acceptable surfactant. More preferably, the first layer comprises at least 2% by weight of a pharmaceutically acceptable surfactant. Highly preferably, the first layer comprises at least 5% by weight of a pharmaceutically acceptable surfactant. In one embodiment, the first layer comprise from 4% to 20% by weight of a pharmaceutically acceptable surfactant. In another embodiment, the first layer comprise from 5% to 10% by weight of a pharmaceutically acceptable surfactant.

Lubricants that are suitable for use in the first layer include, but are not limited to, fatty acid salts (e.g., stearic acid salts such as calcium stearate, magnesium stearate, aluminum stearate, zinc stearate, potassium stearate or sodium stearate); sodium stearyl fumarate or alike; or lauryl sulfate salts (e.g., sodium lauryl sulfate or magnesium lauryl sulfate). Preferred lubricants are solid powder at room temperature, such as sodium stearyl fumarate.

In one embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.7 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.7 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.7 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.6 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.6 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.6 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.5 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.5 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.5 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.5 g, and the first layer is at least 1.1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.5 g, and the first layer is at least 1.1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.5 g, and the first layer is at least 1.1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer).

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.6 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.6 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.6 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.6 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.6 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.6 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.6 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.6 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.6 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 200 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 50 mg or less ritonavir (e.g., 50 mg ritonavir), and at least 50% by weight of the hydrophilic polymer (e.g., from 65 to 75% by weight of the hydrophilic polymer). The first layer is also preferably at least 1.1 g.

In one embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 180 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 45 mg or less ritonavir (e.g., 45 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In one embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.5 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is from 1.2 to 1.4 g (e.g., the tablet is 1.3 g), and the first layer is at least 0.9 g (e.g., the first layer is 1 g). Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.4 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 170 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 42.5 mg or less ritonavir (e.g., 42.5 mg ritonavir), and at least 50% by weight of the hydrophilic polymer (e.g., from 65 to 75% by weight of the hydrophilic polymer). The first layer is also preferably at least 1 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In one embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.5 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.5 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.5 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.4 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.4 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.4 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.3 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.3 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.3 g, and the first layer is at least 0.8 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In still another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.4 g, and the first layer is at least 0.9 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.5% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and no more than 0.2% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

In yet another embodiment, a tablet dosage form of the invention comprises a first layer and a second layer, wherein the first layer comprises a pharmaceutically acceptable hydrophilic polymer, a pharmaceutically acceptable surfactant, ritonavir, 160 mg lopinavir, and 0.1% by weight of one or more lubricants, and wherein the second layer comprises another therapeutic agent (e.g., 75 mg lamivudine). The tablet is no more than 1.4 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The lubricant(s), if any, is preferably sodium stearyl fumarate. The first layer preferably comprises 40 mg or less ritonavir (e.g., 40 mg ritonavir), and at least 50% by weight of the hydrophilic polymer (e.g., from 65 to 75% by weight of the hydrophilic polymer). The first layer is also preferably at least 0.9 g. Preferably, the second layer comprises 75 mg lamivudine; and the tablet dosage form is bioequivalent to a combination of a Kaletra tablet (200 mg lopinavir and 50 mg ritonavir per tablet) and one half of an Epivir tablet (150 mg lamivudine per tablet) with respect to lopinavir and lamivudine, or two tablet dosage forms of this embodiment are bioequivalent to a combination of two Kaletra tablets (200 mg lopinavir and 50 mg ritonavir per tablet) and one Epivir tablet (150 mg lamivudine per tablet) with respect to lopinavir and lamivudine.

The first and second layers of a tablet dosage form of the invention can be prepared by a variety of techniques such as, without limitation, granulation (e.g., wet or dry granulation), blending, melt-extrusion, spray-drying, co-precipitation, freeze drying, or other solvent evaporation or solid dispersion techniques, with melt-extrusion and spray-drying being preferred for the preparation of the first layer.

Melt-extrusion and spray-drying are non-limiting examples that are suitable for making solid dispersions. Powdery solid dispersions can be, for example, pre-compressed to form the first layer of a tablet dosage form of the invention. Non-powdery solid dispersions can be, for example, milled or ground to small particles before being pre-compressed. Additional ingredients may be mixed with the solid dispersions before grinding and/or pre-compression.

The melt-extrusion process typically comprises the steps of preparing a melt which includes the active ingredient(s) (e.g., lopinavir and/or ritonavir), a pharmaceutically acceptable hydrophilic polymer described above, and preferably a pharmaceutically acceptable surfactant described above, and then cooling the melt until it solidifies. Melting often involves a transition into a liquid or rubbery state in which it is possible for one component to get dissolved or embedded, preferably homogeneously dissolved or embedded, in the other component or components such as the pharmaceutically acceptable hydrophilic polymer. In many cases, the polymer component(s) will melt and the other components including the active ingredient(s) and surfactant will dissolve in the melt thereby forming a solution. In such a case, the polymer functions as a solvent. Melting often involves heating above the softening point of the polymer. The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredient(s) efficiently. In addition, it may be convenient first to melt the polymer and then to mix in and homogenize the active ingredient(s). In one example, all materials except the surfactant are blended and fed into an extruder, while the surfactant is molten externally and pumped in during extrusion.

The melt temperature may range, for example, from 70 to 250° C., preferably from 80 to 180° C., most preferred from 100 to 140° C. Various additives may be included in the melt, for example, flow regulators such as colloidal silica; lubricants; fillers; disintegrants; plasticizers; or stabilizers such as antioxidants, light stabilizers, radical scavengers, or stabilizers against microbial attack.

In another example, the melt comprises lopinavir, ritonavir or preferably both, as well as a pharmaceutically acceptable hydrophilic polymer described above; and the melt temperature is in the range of from 100 to 170° C., preferably from 120 to 150° C., and highly preferably from 135 to 140° C. The melt can also include a pharmaceutically acceptable surfactant described above.

In still another example, the melt comprises lopinavir, ritonavir, and a pharmaceutically acceptable polymer described above. The melt can also include a pharmaceutically acceptable surfactant described above. The melt temperature can be in the range of from 100 to 170° C., preferably from 120 to 150° C., and highly preferably from 135 to 140° C.

To start a melt-extrusion process, the active ingredient(s) (e.g., lopinavir and/or ritonavir) can be employed in their solid forms, such as their respective crystalline forms. The active ingredient(s) can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

The melting and/or mixing can take place in an apparatus customary for this purpose. Particularly suitable ones are extruders or kneaders. Suitable extruders include single screw extruders, intermeshing screw extruders or multiscrew extruders, preferably twin screw extruders, which can be corotating or counterrotating and, optionally, be equipped with kneading disks. It will be appreciated that the working temperatures will be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt can range from thin to pasty to viscous. Shaping of the extrudate can be conveniently carried out, for example, by a calender with two counter-rotating rollers with mutually matching depressions on their surface. The extrudate can be cooled and allow to solidify. The extrudate can also be cut into pieces, either before (hot-cut) or after solidification (cold-cut).

The solidified extrusion product can be further milled, ground or otherwise reduced to granules. The solidified extrudate, as well as each granule produced, comprises a solid dispersion, preferably a solid solution or glassy solution, of the active ingredient(s) in a matrix comprised of the hydrophilic polymer and optionally the surfactant. Where the granules do not contain any surfactant, a pharmaceutically acceptable surfactant described above can be, for example, added to and blended with the granules. The extrusion product can also be blended with other active ingredient(s) and/or additive(s) before being milled or ground to granules. The granules can be, for example, further processed and pre-compressed to form the first layer of a tablet dosage form of the invention. The second layer material may then be compressed with the first layer to form a bilayer tablet.

In some cases, direct-shaping techniques such as injection moulding can be used in combination with melt extrusion to prepare the first layer of a tablet dosage form of the invention.

In one example, copovidone and one or more surfactants are mixed and granulated, followed by the addition of aerosil and the active ingredient(s) (e.g., lopinavir, ritonavir, or preferably a combination of lopinavir and ritonavir). The mixture, which may contain for example at least 5% by weight of the active ingredient(s) is then milled. The mixture is then subject to extrusion, and the extrudate thus produced can be milled and sieved for further processing for the preparation of the first layer. Surfactant(s) employed in this example can also be added through liquid dosing during extrusion.

The approach of solvent evaporation, via spray-drying, may provide the advantage of allowing for processability at lower temperatures, if needed, and allow for other modifications to the process in order to further improve powder properties. In many cases, the spray-dried powder can then be formulated further, if needed, and then pre-compressed to form the first layer of a tablet dosage form of the invention.

Exemplary spray-drying processes and spray-drying equipment are described in K. Masters, Spray Drying Handbook (Halstead Press, New York, 4^{th} ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus. This help to ensure that the dried droplets are essentially solid and can form a fine powder and do not stick to the apparatus wall. The spray-dried product can be collected by removing the material manually, pneumatically, mechanically or by other suitable means. The actual length of time to achieve the preferred level of dryness can depend on the size of the droplets, the formulation, and spray dryer operation. Following the solidification, the solid powder may stay in the spray drying chamber for additional time (e.g., 5-60 seconds) to further evaporate solvent from the solid powder. The final solvent content in the solid dispersion as it exits the dryer is preferably at a sufficiently low level so as to improve the stability of the final product. For instance, the residual solvent content of the spray-dried powder can be less than 2% by weight. Highly preferably, the residual solvent content is within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other processes.

Like the solid extrudate described above, the spray dried product can contain a solid dispersion, preferably a solid solution and more preferably a glassy solution, of the active ingredient(s) in a matrix comprised of a pharmaceutically acceptable hydrophilic polymer described above and optionally a pharmaceutically acceptable surfactant described above. Where the spray dried product does not contain any surfactant, a pharmaceutically acceptable surfactant described above may be optionally added to and blended with the spray-dried product before further processing.

Before feeding into a spray dryer, the active ingredient(s) (e.g., lopinavir, ritonavir, or preferably a combination of ritonavir and lopinavir), a pharmaceutically acceptable hydrophilic polymer described above, and optionally a pharmaceutically acceptable surfactant described above can be dissolved in a solvent. Suitable solvents include, but are not limited to, water, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

A solid dispersion (preferably a solid solution or a glassy solution) prepared by melt-extrusion, spray-drying or other techniques may include both lopinavir and ritonavir. Ritonavir solid dispersion and lopinavir solid dispersion can also be separately prepared as described above, optionally milled, blended, and then pre-compressed into the first layer. In one embodiment, ritonavir solid dispersion described above and lopinavir solid dispersion described above are separately prepared, and compressed into two different layers in the first layer of a tablet dosage form of the invention.

The first layer of a tablet dosage form of the invention can also comprise one or more one additives, such as flow regulators, binders, lubricants, fillers, disintegrants, or plasticizers. These additives can be, for example, mixed with milled or powdery solid dispersions before pre-compression. These additives can also be, for example, mixed with solid dispersions before milling. Disintegrants may promote a rapid disintegration of a tablet in the stomach and keep the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell® or Emcocel®), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil®). Non-limiting examples of suitable lubricants include those described above, such as magnesium and calcium stearates, sodium stearyl fumarate, and the like.

The second layer of a tablet dosage form of the invention can be prepared using techniques that are suitable for the formulation of the other therapeutic agent comprised in the second layer. Non-limiting examples of these techniques include blending, dry granulation, wet granulation, or solid dispersion. For instance, the other therapeutic agent (e.g., lamivudine) can be blended with suitable excipients, milled, and then compressed against the first layer to form a bilayer tablet core. Other additives described above may also be included in the second layer.

Preferably, the weight ratio of the second layer to the first layer is no more than 1:3, such as no more than 1:4, 1:5, or 1:6. Techniques suitable for compressing two layers together in a tablet are known in the art. In many cases, the first layer can be pre-compressed before the compression of the second layer.

The present invention unexpected found that it was difficult to compress the second layer against the first layer to form a stable bilayer tablet core when the weight ratio of the second layer to the first layer is substantially less than 1:1. The present invention also unexpected found that by reducing the amount of lubricant(s) in the first layer, the feasibility of compressing the first and second layers to form a stable bilayer core can be significantly improved. Preferably, the compression force used in compressing the first layer and the second layer together is no more than 30 kN. More preferably, the compression force is no more than 25 kN. Highly preferably, the compression force is 23 kN.

In order to facilitate the intake of a tablet dosage form, it is advantageous to give the tablet an appropriate shape. Large tablets that can be swallowed comfortably are therefore preferably elongated rather than round in shape.

A film coat on the tablet core further contributes to the ease with which it can be swallowed. A film coat also improves taste and provides an elegant appearance. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. polysorbates, and optionally a pigment, e.g. titanium dioxide or iron oxides. The film-coating may also comprise talc as anti-adhesive. Preferably, the film coat accounts for less than 5% by weight of a tablet dosage form of the present invention. In one embodiment, a tablet dosage form of the invention (e.g., with a bilayer tablet core described herein) is coated with Opadry® HPMC based coating solution.

Various other additives may also be used in preparing a tablet dosage form of the invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack.

Tablet dosage forms according to certain embodiments of the present invention may contain more than two layers. In one embodiment, the layer comprising the other therapeutic agent is sandwiched between a ritonavir layer and a lopinavir layer, wherein the ritonavir layer comprises a ritonavir solid dispersion described above and the lopinavir layer comprises a lopinavir solid dispersion described above. In another embodiment, the layer comprising the other therapeutic agent is sandwiched between two layers, wherein each of the two layers has the same or similar composition as a first layer described herein.

In another aspect, the present invention features tablet dosage forms suitable for pediatric uses. Each tablet dosage form of the invention described hereinabove has a corresponding pediatric tablet dosage form, wherein the pediatric tablet has the same active ingredients and excipients as the regular tablet, except that each component in the pediatric tablet is only one half of the amount of the same component in the regular tablet. For instance, a pediatric tablet dosage form of the invention can comprise a first layer and a second layer, wherein the first layer comprises (i) from 80 to 100 mg lopinavir (e.g., 100 mg lopinavir, or 90 mg lopinavir, or 85 mg lopinavir, or 80 mg lopinavir), (ii) ritonavir, (iii) a pharmaceutically acceptable hydrophilic polymer described above, (iv) a pharmaceutically acceptable surfactant described above, and (v) no more than 0.9% by weight of one or more lubricants; and the second layer comprises another therapeutic agent (e.g., 37.5 mg lamivudine). Preferably, the first layer contains no more than 0.5% by weight of any lubricants. More preferably, the first layer contains no more than 0.2% by weight of any lubricants. Highly preferably, the first layer contains no more than 0.1% by weight of any lubricants.

The weight ratio of lopinavir to ritonavir in the first layer in a pediatric tablet dosage form of the invention can be, for example and without limitation, 4:1. For instance, the first layer can comprise 100 mg lopinavir and 25 mg ritonavir, or 90 mg lopinavir and 22.5 mg ritonavir, or 85 mg lopinavir and 21.25 mg ritonavir, or 80 mg lopinavir and 20 mg ritonavir.

The weight ratio of the second layer to the first layer in a pediatric tablet dosage form of the invention can be, for example and without limitation, no more than 1:4. Preferably, the weight ratio of the second layer to the first layer is no more than 1:5; for example, the weight ratio of the second layer to the first layer can be 1:5, 1:6 or less. Also preferably, the first layer comprises at least 60% weight of the tablet dosage form. More preferably, the first layer comprises at least 70% weight of the tablet dosage form. Highly preferably, the first layer comprises at least 80% weight of the tablet dosage form.

A pediatric tablet dosage form of the invention can be, for example and without limitation, from 0.55 g to 0.85 g, and the first layer can be, for example and without limitation, from 0.45 g to 0.65 g. For instance, the tablet dosage form can be from 0.55 g to 0.75 g, and the first layer is from 0.45 g to 0.65 g. For instance, the tablet dosage form can be from 0.6 g to 0.7 g, and the first layer is from 0.5 g to 0.6 g. In one example, the tablet dosage form is no more than 0.85 g, and the first layer is at least 0.5 g. In another example, the tablet dosage form can be no more than 0.8 g, and the first layer is at least 0.5 g. In another yet example, the tablet dosage form can be no more than 0.75 g, and the first layer is at least 0.5 g. In yet another example, the tablet dosage form is no more than 0.7 g, and the first layer is at least 0.5 g. In yet another example, the tablet dosage form is no more than 0.85 g, and the first layer is at least 0.55 g. In yet another example, the tablet dosage form is no more than 0.8 g, and the first layer is at least 0.55 g. In yet another example, the tablet dosage form is no more than 0.75 g, and the first layer is at least 0.55 g. In yet another example, the tablet dosage form is no more than 0.7 g, and the first layer is at least 0.55 g. In yet another example, the tablet dosage form is no more than 0.85 g, and the first layer is at least 0.6 g. In yet another example, the tablet dosage form is no more than 0.8 g, and the first layer is at least 0.6 g. In yet another example, the tablet dosage form is no more than 0.75 g, and the first layer is at least 0.6 g. In yet another example, the tablet dosage form is no more than 0.85 g, and the first layer is from 0.5 g to 0.65 g. In another example, the tablet dosage form is no more than 0.85 g, and the first layer is at from 0.5 g to 0.6 g. In yet another example, the tablet dosage form is no more than 0.85 g, and the first layer is from 0.5 g to 0.55 g. In yet another example, the tablet dosage form is no more than 0.85 g, and the first layer is from 0.55 g to 0.65 g. In yet another example, the tablet dosage form is no more than 0.85 g, and the first layer is from 0.55 g to 0.6 g. In yet another example, the tablet dosage form is no more than 0.8 g, and the first layer is from 0.5 g to 0.65 g. In another example, the tablet dosage form is no more than 0.8 g, and the first layer is at from 0.5 g to 0.6 g. In yet another example, the tablet dosage form is no more than 0.8 g, and the first layer is from 0.5 g to 0.55 g. In yet another example, the tablet dosage form is no more than 0.8 g, and the first layer is from 0.55 g to 0.65 g. In yet another example, the tablet dosage form is no more than 0.8 g, and the first layer is from 0.55 g to 0.6 g. In yet another example, the tablet dosage form is no more than 0.75 g, and the first layer is from 0.5 g to 0.65 g. In another example, the tablet dosage form is no more than 0.75 g, and the first layer is at from 0.5 g to 0.6 g. In yet another example, the tablet dosage form is no more than 0.75 g, and the first layer is from 0.5 g to 0.55 g. In yet another example, the tablet dosage form is no more than 0.75 g, and the first layer is from 0.55 g to 0.65 g. In yet another example, the tablet dosage form is no more than 0.75 g, and the first layer is from 0.55 g to 0.6 g.

The first layer in a pediatric tablet dosage form of the invention preferably includes a solid dispersion comprising ritonavir, lopinavir, a pharmaceutically acceptable hydrophilic polymer described above, and a pharmaceutically acceptable surfactant described above. More preferably, the first layer comprises a solid solution comprising ritonavir, lopinavir, a pharmaceutically acceptable hydrophilic polymer described above, and a pharmaceutically acceptable surfactant described above. Highly preferably, the first layer comprises a glassy solution comprising ritonavir, lopinavir, a pharmaceutically acceptable hydrophilic polymer described above, and a pharmaceutically acceptable surfactant described above.

In a pediatric tablet dosage form of the invention, lopinavir and ritonavir can also be formulated, for example, in different solid dispersions and then mixed and included in the first layer. In one example, the first layer comprises a first and second solid dispersions, wherein the first solid dispersion comprises lopinavir and a first pharmaceutically acceptable hydrophilic polymer, and the second solid dispersion comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid dispersions, wherein the first solid dispersion comprises lopinavir, a first pharmaceutically acceptable hydrophilic polymer, and a first pharmaceutically acceptable surfactant, and the second solid dispersion comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In yet another example, the first layer comprises a first and second solid solutions, wherein the first solid solution comprises lopinavir and a first pharmaceutically acceptable hydrophilic polymer, and the second solid solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid solutions, wherein the first solid solution comprises lopinavir, a first pharmaceutically acceptable hydrophilic polymer, and a first pharmaceutically acceptable surfactant, and the second solid solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In still another example, the first layer comprises a first and second glassy solutions, wherein the first glassy solution comprises lopinavir and a first pharmaceutically acceptable hydrophilic polymer, and the second glassy solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second glassy solutions, wherein the first glassy solution comprises lopinavir, a first pharmaceutically acceptable hydrophilic polymer, and a first pharmaceutically acceptable surfactant, and the second glassy solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

Any other therapeutic agents that can be included in the second layer of a regular tablet dosage form can be included in the second layer of a pediatric tablet dosage form of the invention. In one embodiment, the second layer of a pediatric tablet dosage form comprises 37.5 mg lamivudine. In another embodiment, the second layer of a pediatric tablet dosage form comprises 37.5 mg lamivudine and 75 mg zidovudine. In yet another embodiment, the second layer of a pediatric tablet dosage form comprises 100 mg raltegravir potassium.

In yet another aspect, the present invention features methods of using a tablet dosage form of the invention to treat HIV infection. The methods comprise administering a tablet dosage form of the invention to a patient in need thereof. Any tablet dosage form described herein (including a pediatric tablet dosage) can be used in a method of the invention.

As another aspect of the present invention, a solid pharmaceutical dosage form is provided which comprises (a) lopinavir or a pharmaceutically acceptable salt thereof, (b) ritonavir or a pharmaceutically acceptable salt thereof; and (c) lamivudine or a pharmaceutically acceptable salt thereof. The solid pharmaceutical dosage form can be a bilayer tablet comprising at least a first layer and a second layer, wherein the first layer comprises ritonavir (or a pharmaceutically acceptable salt thereof) and lopinavir (or a pharmaceutically acceptable salt thereof), and the second layer comprises lamivudine (or a pharmaceutically acceptable salt thereof).

As another aspect of the present invention, a solid pharmaceutical dosage form or pharmaceutical preparation is provided which comprises (a) 200 mg of lopinavir or a pharmaceutically acceptable salt thereof; (b) 50 mg of ritonavir or a pharmaceutically acceptable salt thereof, (c) from 55% to 75% by weight, alternatively 68% to 72% by weight, of N-vinyl pyrrolidone/vinyl acetate copolymer 60:40 or another pharmaceutically acceptable water-soluble polymer, based on total weight of the dosage form; (d) from 4% to 10% by weight of sorbitan monolaurate or another pharmaceutically acceptable surfactant, based on total weight of the dosage form; (e) from 0.05% to less than 1% by weight, alternatively less than 0.8% by weight, alternatively less than 0.6% by weight, alternatively less than 0.4% by weight, alternatively 0.2% by weight or less, alternatively from 0.1% to 0.2% by weight, of sodium stearyl fumarate or another lubricant, based on total weight of the dosage form; and (f) from 0.01% to 1.6% by weight of colloidal silicon dioxide, alternatively less than 1.6% by weight of colloidal silicon dioxide or another flow regulator, alternatively 1.4% by weight or less, alternatively 1.2% by weight or less, alternatively 1.1% by weight or less, 1% by weight or less, relative to total weight of the first layer. In some embodiments, the weight percentages are based on the weight of an uncoated tablet. In other embodiments, the dosage form comprises colloidal silicon dioxide in an amount that is from 0.9% to 1.3% by weight, relative to total weight of the dosage form.

As another aspect of the present invention, a solid pharmaceutical dosage form or pharmaceutical preparation is provided which comprises (a) 180 mg of lopinavir or a pharmaceutically acceptable salt thereof; (b) 45 mg of ritonavir or a pharmaceutically acceptable salt thereof; (c) from 55% to 75% by weight, alternatively 68% to 72% by weight, of N-vinyl pyrrolidone/vinyl acetate copolymer 60:40 or another pharmaceutically acceptable water-soluble polymer, based on total weight of the dosage form; (d) from 4% to 10% by weight of sorbitan monolaurate or another pharmaceutically acceptable surfactant, based on total weight of the dosage form; (e) from 0.05% to less than 1% by weight, alternatively less than 0.8% by weight, alternatively less than 0.6% by weight, alternatively less than 0.4% by weight, alternatively 0.2% by weight or less, alternatively from 0.1% to 0.2% by weight, of sodium stearyl fumarate or another lubricant, based on total weight of the dosage form; and (0 from 0.01% to 1.6% by weight of colloidal silicon dioxide, alternatively less than 1.6% by weight of colloidal silicon dioxide or another flow regulator, alternatively 1.4% by weight or less, alternatively 1.2% by weight or less, alternatively 1.1% by weight or less, 1% by weight or less, relative to total weight of the first layer. In some embodiments, the weight percentages are based on the weight of an uncoated tablet. In other embodiments, the dosage form comprises colloidal silicon dioxide in an amount that is from 0.9% to 1.3% by weight, relative to total weight of the dosage form.

As another aspect of the present invention, a solid pharmaceutical dosage form or pharmaceutical preparation is provided which comprises (a) 170 mg of lopinavir or a pharmaceutically acceptable salt thereof; (b) 42.5 mg of ritonavir or a pharmaceutically acceptable salt thereof; (c) from 55% to 75% by weight, alternatively 68% to 72% by weight, of N-vinyl pyrrolidone/vinyl acetate copolymer 60:40 or another pharmaceutically acceptable water-soluble polymer, based on total weight of the dosage form; (d) from 4% to 10% by weight of sorbitan monolaurate or another pharmaceutically acceptable surfactant, based on total weight of the dosage form; (e) from 0.05% to less than 1% by weight, alternatively less than 0.8% by weight, alternatively less than 0.6% by weight, alternatively less than 0.4% by weight, alternatively 0.2% by weight or less, alternatively from 0.1% to 0.2% by weight, of sodium stearyl fumarate or another lubricant, based on total weight of the dosage form; and (f) from 0.01% to 1.6% by weight of colloidal silicon dioxide, alternatively less than 1.6% by weight of colloidal silicon dioxide or another flow regulator, alternatively 1.4% by weight or less, alternatively 1.2% by weight or less, alternatively 1.1% by weight or less, 1% by weight or less, relative to total weight of the first layer. In some embodiments, the weight percentages are based on the weight of an uncoated tablet. In other embodiments, the dosage form comprises colloidal silicon dioxide in an amount that is from 0.9% to 1.3% by weight, relative to total weight of the dosage form.

As another aspect of the present invention, a solid pharmaceutical dosage form or pharmaceutical preparation is provided which comprises (a) 160 mg of lopinavir or a pharmaceutically acceptable salt thereof; (b) 40 mg of ritonavir or a pharmaceutically acceptable salt thereof; (c) from 55% to 75% by weight, alternatively 68% to 72% by weight, of N-vinyl pyrrolidone/vinyl acetate copolymer 60:40 or another pharmaceutically acceptable water-soluble polymer, based on total weight of the dosage form; (d) from 4% to 10% by weight of sorbitan monolaurate or another pharmaceutically acceptable surfactant, based on total weight of the dosage form; (e) from 0.05% to less than 1% by weight, alternatively less than 0.8% by weight, alternatively less than 0.6% by weight, alternatively less than 0.4% by weight, alternatively 0.2% by weight or less, alternatively from 0.1% to 0.2% by weight, of sodium stearyl fumarate or another lubricant, based on total weight of the dosage form; and (0 from 0.01% to 1.6% by weight of colloidal silicon dioxide, alternatively less than 1.6% by weight of colloidal silicon dioxide or another flow regulator, alternatively 1.4% by weight or less, alternatively 1.2% by weight or less, alternatively 1.1% by weight or less, 1% by weight or less, relative to total weight of the first layer. In some embodiments, the weight percentages are based on the weight of an uncoated tablet. In other embodiments, the dosage form comprises colloidal silicon dioxide in an amount that is from 0.9% to 1.3% by weight, relative to total weight of the dosage form.

As yet another aspect, a method of preparing a solid pharmaceutical dosage form comprising lopinavir, ritonavir, and lamivudine, or a pharmaceutically acceptable salt of one or more of the foregoing, is provided. The method comprises preparing a homogeneous melt of ritonavir or pharmaceutically acceptable salt thereof, lopinavir or pharmaceutically acceptable salt thereof, a pharmaceutically acceptable water-soluble polymer(s), a pharmaceutically acceptable surfactant(s) and a flow regulator(s). The method also comprises extruding the melt to obtain an extrudate, with optional milling of the extrudate. The method also comprises mixing the extrudate with a flow regulator and a lubricant to form a first pharmaceutical preparation, and adding the first pharmaceutical preparation to a bilayer tablet press. In some embodiments, the amounts of flow regulator and/or lubricant mixed with the extrudate are surprisingly low. The method also comprises adding a second pharmaceutical preparation to the bilayer tablet press, wherein the second pharmaceutical preparation comprises lamivudine or pharmaceutically acceptable salt thereof. The method also comprises compressing the first pharmaceutical preparation and the second pharmaceutical preparation together to form a bilayer tablet. Preferably, the first pharmaceutical preparation is subjected to an initial compression step before that preparation is compressed with the second pharmaceutical preparation.

As another aspect of the present invention, methods of treating an HIV infection are provided. The methods comprise administering one of the solid pharmaceutical dosage forms described herein to a mammal in need of such treatment.

In one aspect, a solid pharmaceutical dosage form of the invention can be a bilayer tablet, a multilayer tablet, a capsule, a monothilic matrix or another dosage form. Preferably, the solid pharmaceutical dosage form is a bilayer tablet comprising at least a first layer and a second layer, wherein the first layer comprises ritonavir (or a pharmaceutically acceptable salt thereof) and lopinavir (or a pharmaceutically acceptable salt thereof), and the second layer comprises lamivudine (or a pharmaceutically acceptable salt thereof). In some embodiments, the first layer comprises at least 75% by weight of the bilayer tablet, alternatively at least 80% by weight of the bilayer tablet, alternatively at least 85% by weight of the bilayer tablet. As a non-limiting example, a bilayer tablet of the invention that contains lopinavir and ritonavir in a first layer and lamivudine in a second layer can be prepared by providing a second layer that is smaller than the first layer (for example, the second layer is 25% or less, alternatively 20% or less, alternatively 15% or less by weight of the bilayer tablet).

In many embodiments, for the second layer to be relatively smaller yet contain a desired dose amount of lamivudine, a relatively high percentage or loading of lamivudine can be included in the second layer. The solid pharmaceutical dosage form can be a solid oral dosage form which is administered orally to a patient, having a suitable size, shape and/or coating to facilitate oral administration.

As an aspect of the present invention, a solid pharmaceutical dosage form or a pharmaceutical preparation comprising lopinavir and ritonavir is provided which has a far lower amount of certain excipients than current Kaletra tablets. For instance, a novel solid pharmaceutical dosage form (or pharmaceutical preparation) can have less than 1% by weight of a lubricant such as sodium stearyl fumarate, based on the weight of the uncoated tablet. Alternatively or additionally, a solid pharmaceutical dosage form can have less than 1.6% by weight of a flow regulator such as colloidal silicon dioxide, based on the weight of the uncoated tablet. Alternatively, the weight percentage of the sodium stearyl fumarate and silicon dioxide is based on the weight of a coated tablet or other dosage form. In some embodiments, a mixture comprising lopinavir, ritonavir, a water soluble polymer, a surfactant, and colloidal silicon dioxide is extruded, and the extrudate is then mixed with an amount of sodium stearyl fumarate that is 1% by weight or less, alternatively 0.8% by weight or less, alternatively 0.7% by weight or less, alternatively 0.6% by weight or less, alternatively 0.4% by weight or less, alternatively 0.2% by weight or less, preferably 0.1% by weight or from 0.05% to 0.15% by weight, relative to total weight of the extrudate, and an amount of colloidal silicon dioxide that is 0.6% by weight or less, alternatively 0.4% by weight or less, alternatively 0.2% by weight or less, alternatively 0.1% by weight or less, alternatively 0.08% by weight or less, preferably from 0.06% to 0.07% by weight, relative to total weight of the extrudate. In other embodiments, the extrudate is then mixed with an amount of sodium stearyl fumarate that is from 0.6% to 0.7% by weight, relative to total weight of the extrudate. Methods of preparing such a solid pharmaceutical dosage form comprising mixing an extrudate comprising lopinavir, ritonavir and other excipients with the foregoing amounts of sodium stearyl fumarate and/or colloidal silicon dioxide are also contemplated.

A pharmaceutical preparation, as used herein, contains an anti-HIV drug and one or more pharmaceutically acceptable excipients. The term may refer to a solid pharmaceutical dosage form, or a portion of a dosage form, or a mixture of powders to be compressed into a dosage form. For example, a pharmaceutical preparation may form, for example, a layer of a multilayer tablet (e.g., a layer of a bilayer tablet), or it may be suitable for use as a pharmaceutical dosage form by itself. A pharmaceutical preparation may also be an uncoated dosage form, optional fully or partially covered by a coating, or may refer to the coated dosage form.

In some embodiments of the solid dosage forms of this aspect of the invention, the pharmaceutical dosage form comprises two layers of anti-HIV agents. The first layer comprises at least a combination of ritonavir and lopinavir. Lopinavir in the first layer of the pharmaceutical dosage form is present in a suitable dose amount, for example 200, 180, 170 or 160 mg, alternatively 100, 90, 85 or 80 mg in a pediatric dosage form; and ritonavir in the first layer of the pharmaceutical dosage form is present in a suitable dose amount, for example, one-fourth of the amount of lopinavir, such as 50, 45, 42.5 or 40 mg, respectively, alternatively 25, 22.5, 21.25 or 20 mg in a pediatric dosage form, respectively. The second layer of the pharmaceutical dosage form comprises lamivudine. Lamivudine in the second layer of the pharmaceutical dosage form is present in an amount from 3% to 10% by weight of the total dosage form (preferably from 4% to 7% by weight of the total dosage form). In some embodiments, the solid pharmaceutical dosage form contains lopinavir in an amount of 200 mg; ritonavir in an amount of 50 mg; and lamivudine in an amount of 75 mg. In some embodiments, the solid pharmaceutical dosage form contains lopinavir in an amount of 180 mg; ritonavir in an amount of 45 mg; and lamivudine in an amount of 75 mg. In some embodiments, the solid pharmaceutical dosage form contains lopinavir in an amount of 170 mg; ritonavir in an amount of 42.5 mg; and lamivudine in an amount of 75 mg. In some embodiments, the solid pharmaceutical dosage form contains lopinavir in an amount of 160 mg; ritonavir in an amount of 40 mg; and lamivudine in an amount of 75 mg. In some embodiments, the solid pharmaceutical dosage form contains lopinavir in an amount of 100 mg; ritonavir in an amount of 25; and lamivudine in an amount of 37.5 mg. In some embodiments, the solid pharmaceutical dosage form contains lopinavir in an amount of 90 mg; ritonavir in an amount of 22.5; and lamivudine in an amount of 37.5 mg. In some embodiments, the solid pharmaceutical dosage form contains lopinavir in an amount of 85 mg; ritonavir in an amount of 21.25; and lamivudine in an amount of 37.5 mg. In some embodiments, the solid pharmaceutical dosage form contains lopinavir in an amount of 80 mg; ritonavir in an amount of 20; and lamivudine in an amount of 37.5 mg.

In some embodiments, a solid pharmaceutical dosage form or a tablet dosage form of this aspect of the invention consists essentially of ritonavir, lopinavir, and lamivudine (or a pharmaceutically acceptable salt of one or more of the foregoing) and one or more excipients. In some embodiments, the dosage forms do not contain any active ingredient other than ritonavir, lopinavir, and lamivudine (or their salts).

The solid pharmaceutical dosage forms of this aspect of the invention can include various excipients. For example, the dosage forms of the invention may contain other pharmaceutical additives, such as pharmaceutically acceptable water-soluble polymers, pharmaceutically acceptable surfactants, flow regulators, lubricants, bulking agents (fillers) and disintegrants. When the solid pharmaceutical dosage form is a bilayer tablet, the individual layers may have the same or different excipients, in the same or different weight percentages. For example, in an exemplary bilayer tablet, a layer containing lopinavir and ritonavir may include a pharmaceutically acceptable water-soluble polymer, a pharmaceutically acceptable surfactant, a flow regulator, a bulking agent and a glidant, and a layer containing lamivudine may include a pharmaceutically acceptable water-soluble polymer, a flow regulator, a bulking agent and a glidant, wherein the specific excipients (for example, the flow regulator and glidant) may be the same in both layers, while other excipients (for example, the pharmaceutically acceptable water-soluble polymer and bulking agent) may be different in the different layers.

In some embodiments, a solid pharmaceutical dosage form or a tablet dosage form of this aspect of the invention has a first layer containing lopinavir and ritonavir, and the first layer further comprises from 55% to 75% by weight, alternatively from 68% to 72% by weight, of at least one pharmaceutically acceptable water-soluble polymer, based on total weight of the first layer; from 4% to 10% by weight of at least one pharmaceutically acceptable surfactant; from 0.05% to 1% by weight of at least one lubricant (for example, sodium stearyl fumarate), based on total weight of the first layer; and from 0.01% to 3% by weight of at least one flow regulator (for example, colloidal silicon dioxide), based on total weight of the first layer. In preferred embodiments, the first layer comprises less than 1% by weight of a lubricant (for example, sodium stearyl fumarate), alternatively 0.8% by weight or less, alternatively 0.6% by weight or less, alternatively 0.4% by weight or less, alternatively 0.2% by weight or less, preferably 0.1% by weight, alternatively from 0.1% to 0.2% by weight, relative to total weight of the first layer. Additionally or alternatively, in some embodiments, the first layer comprises less than 1.6% by weight of colloidal silicon dioxide, alternatively 1.5% by weight or less, alternatively 1.2% by weight or less, alternatively 1.1% by weight or less, relative to total weight of the first layer. In some preferred embodiments of the solid pharmaceutical dosage form, the first layer comprises 55% to 75% by weight, alternatively from 68% to 72% by weight, of N-vinyl pyrrolidone/vinyl acetate copolymer 60:40, based on total weight of the first layer; from 3% to 10% by weight of sorbitan monolaurate, based on total weight of the first layer; from 0.05% to 0.15% by weight of sodium stearyl fumarate, based on total weight of the first layer; and from 1% to 1.2% by weight of colloidal silicon dioxide, based on total weight of the first layer.

In some embodiments, a solid pharmaceutical dosage form or a tablet dosage form of this aspect of the invention has a second layer which comprises from 21.4% to 50% by weight of lamivudine, alternatively from 30% to 40% by weight, preferably 35.7% by weight, based on the weight of the second layer. The second layer can also comprise from 0% to 75% by weight of microcrystalline cellulose, based on total weight of the second layer; from 0% to 50% by weight of lactose, based on total weight of the second layer; from 1.0% to 5.0% by weight of sodium starch glycolate, based on total weight of the second layer; from 0.1% to 2.0% by weight of sodium stearyl fumarate, based on total weight of the second layer; from 0.2% to 3.0% by weight of colloidal silicon dioxide, based on total weight of the second layer; and from 0% to 75% by weight of hydroxypropyl cellulose, based on total weight of the second layer. More preferably, the second layer comprises from 60% by weight of microcrystalline cellulose; 3% by weight of sodium starch glycolate; 0.8% by weight of sodium stearyl fumarate; and from 0.2% to 0.4% by weight of colloidal silicon dioxide.

In some embodiments of the solid dosage forms of this aspect of the invention, a pharmaceutical preparation or layer containing lamivudine does not contain, or is essentially free of, any pharmaceutically acceptable water-soluble polymer. Alternatively or additionally, a pharmaceutical preparation or layer containing lamivudine does not contain, or is essentially free of, bulking agent. Preferably, the lamivudine layer is free of lactose.

In yet another aspect, the present disclosure provides a solid oral dosage form comprising at least two layers. At least one of the layers can be a solid dispersion containing at least one anti-HIV agent in at least one pharmaceutically acceptable water-soluble polymer and at least one pharmaceutically acceptable surfactant. In one embodiment, the pharmaceutically acceptable water-soluble polymer has a T_{g} of at least 50° C.

Ritonavir, lopinavir and lamivudine in any desired solid form, including any crystalline form or amorphous form, may be used.

Various methods can be used for manufacturing the solid dosage forms according to this aspect of the invention. As non-limiting examples, these methods comprise the preparation of a solid solution of lopinavir and ritonavir in a matrix of the water-soluble polymer and the surfactant, and shaping into the required tablet form. Alternatively, the solid solution product may be subdivided to granules, e.g. by grinding or milling, and the granules may subsequently be compacted to tablets.

As yet another aspect of the present technology, a method of preparing a solid pharmaceutical dosage form comprising lopinavir, ritonavir, and lamivudine, or a pharmaceutically acceptable salt of one or more of the foregoing, is provided. The method comprises preparing a homogeneous melt of ritonavir or pharmaceutically acceptable salt thereof, lopinavir or pharmaceutically acceptable salt thereof, a water-soluble polymer(s) and a surfactant(s). The method also comprises extruding the melt to obtain an extrudate, with optional milling of the extrudate. The extrudate is mixed with a flow regulator and a lubricant to form a first pharmaceutical preparation, and the first pharmaceutical preparation is added to a bilayer tablet press. The method also comprises adding a second pharmaceutical preparation to the bilayer tablet press, wherein the second pharmaceutical preparation comprises lamivudine or pharmaceutically acceptable salt thereof. The first pharmaceutical preparation and the second pharmaceutical preparation are compressed together to form a bilayer tablet. In some embodiments of the method, the amount of lubricant (for example, sodium stearyl fumarate) is less than 1% by weight, alternatively less than 0.8% by weight, alternatively 0.7% by weight or less, alternatively 0.6% by weight or less, alternatively 0.4% by weight or less, preferably 0.2% by weight or less, based on the total weight of the dosage form. Alternatively or additionally, in some embodiments of the method, the amount of flow regulator (for example, colloidal silicon dioxide) mixed with the extrudate is 0.6% by weight or less, alternatively 0.4% by weight or less, 0.2% by weight or less, 0.1% by weight or less, 0.09% by weight or less, 0.08% by weight or less, most preferably from 0.06% to 0.07% by weight, based on total weight of the extrudate. The method can comprise the step of initially compressing the first pharmaceutical preparation before compressing the second pharmaceutical preparation, wherein the initial compressing step is at a lower pressure than the compression of the first pharmaceutical preparation and second pharmaceutical preparation together. For example, the initial compression of the first preparation can be at a pressure of 1 to 15 kN, preferably 1 to 2 kN, and the later compression to form the tablet can be at a pressure of 20 to 50 kN, such as 20 to 35 kN, and preferably less than 25 kN. The compression pressure may partially depend on tablet size and can be adjusted based on tablet size.

In some embodiments, a bilayer tablet would be formed by compressing a first layer and a second layer on a power-assisted automatic bilayer tablet press. Bilayer tablets may be manufactured via compression of two dry powder blends. For example, blends of the components of each of the two layers may be poured into the hoppers if a rotary bilayer tablet press (for example, a Manesty Model double sided rotary tablet compression machine or a Riva Piccola bilayer press). Alternatively one or both layers may be prepared before the layers are combined to form a bilayer tablet.

The exact dose and frequency of administration depends on the particular condition being treated, the age, weight and general physical condition of the particular patient as well as other medication the individual may be taking, as is well known to those skilled in the art.

In one embodiment, the present invention provides a solid pharmaceutical dosage form comprising ritonavir and lopinavir and lamivudine (or one or more of their pharmaceutically acceptable salts), wherein the dosage form exhibits a suitable in-vitro dissolution profile for each of the anti-HIV agents. The in vitro release profiles can be obtained in any suitable manner, for example by using the USP type 2 dissolution apparatus, at 75 rpm in 900 ml of 0.06M polyoxyethylene 10 lauryl ether (POE10LE) for 120 minutes at 37° C. When measured in that manner, some embodiments of the present solid pharmaceutical dosage forms have an in-vitro release profile of the lamivudine or salt thereof wherein at least 95%, alternatively 100%, is released from the dosage form within ten minutes. Additionally or alternatively, some embodiments have an in-vitro release profile of the lopinavir or salt thereof wherein at least 50% is released from the dosage form within 30 minutes, at least 80% is released from the dosage form within 60 minutes, alternatively within 90%, and 100% is released from the dosage form within 120 minutes. Additionally or alternatively, some embodiments have an in-vitro release profile of the ritonavir or salt thereof wherein at least 40%, alternatively 50%, is released from the dosage form within 30 minutes, at least 80% is released from the dosage form within 60 minutes, alternatively within 90 minutes, and 100% is released from the dosage form within 120 minutes.

As yet another aspect of the present invention, a solid pharmaceutical dosage form is provided which comprises (a) lopinavir or a pharmaceutically acceptable salt thereof; (b) ritonavir or a pharmaceutically acceptable salt thereof; and (c) an additional anti-HIV agent or a pharmaceutically acceptable salt thereof. The solid pharmaceutical dosage form can be a bilayer tablet comprising at least a first layer and a second layer, wherein the first layer comprises the ritonavir or pharmaceutically acceptable salt thereof and the lopinavir or pharmaceutically acceptable salt thereof, and the second layer comprises the additional anti-HIV agent or pharmaceutically acceptable salt thereof.

As yet another aspect, a method of preparing a solid pharmaceutical dosage form comprising lopinavir, ritonavir, and an additional anti-HIV agent, or a pharmaceutically acceptable salt of one or more of the foregoing, is provided. The method comprises preparing a homogeneous melt of ritonavir or pharmaceutically acceptable salt thereof, lopinavir or pharmaceutically acceptable salt thereof, a pharmaceutically acceptable water-soluble polymer(s), a pharmaceutically acceptable surfactant(s) and a flow regulator(s). The method also comprises extruding the melt to obtain an extrudate, with optional milling of the extrudate. The method also comprises mixing the extrudate with a flow regulator and a lubricant to form a first pharmaceutical preparation, and adding the first pharmaceutical preparation to a bilayer tablet press. In some embodiments, the amounts of flow regulator and/or lubricant mixed with the extrudate are surprisingly low. The method also comprises adding a second pharmaceutical preparation to the bilayer tablet press, wherein the second pharmaceutical preparation comprises an additional anti-HIV agent or pharmaceutically acceptable salt thereof. The method also comprises compressing the first pharmaceutical preparation and the second pharmaceutical preparation together to form a bilayer tablet. Preferably, the first pharmaceutical preparation is subjected to an initial compression step before that preparation is compressed with the second pharmaceutical preparation.

Suitable anti-HIV agents to be included in the second layer or the second pharmaceutical preparation include anti-HIV agents that are approved for administration to human subjects having HIV infection as well as anti-HIV agents undergoing development or clinical testing. These anti-HIV agents may be protease inhibitors; nucleoside reverse transcriptase inhibitors (NRTIs); non-nucleoside reverse transcriptase inhibitors (NNRTIs); entry or fusion inhibitors; or integrase inhibitors. In some preferred embodiments, the additional anti-HIV agent is an NRTI. In some preferred embodiments, the additional anti-HIV agent is an NNRTI. In some preferred embodiments, the additional anti-HIV agent is an entry or fusion inhibitor. For example, the anti-HIV agent can be selected from the group consisting of nucleoside reverse transcriptase inhibitors, non-nucleoside reverse transcriptase inhibitors, entry or fusion inhibitors, and mixtures thereof. Non-limiting examples of suitable additional anti-HIV agents are: atazanavir, darunavir, amprenavir, fosamprenavir, indinavir, nelfinavir, saquinavir, tipranavir, lamivudine, abacavir, stavudine, didanosine, zidovudine, emtricitabine, tenofovir, delavirdine, efavirenz, rilpivirine, etravirine, nevirapine, enfuvirtide, maraviroc, raltegravir, as well as pharmaceutically acceptable salts of any of the foregoing anti-HIV agents, or combinations of two or more of the foregoing anti-HIV agents or their salts, such as a combination of emtricitabine and tenofovir.

In one aspect, a solid pharmaceutical dosage form can be a bilayer tablet, a multilayer tablet, a capsule, a monothilic matrix or another dosage form. Preferably, the solid pharmaceutical dosage form is a bilayer tablet comprising at least a first layer and a second layer, wherein the first layer comprises the ritonavir or pharmaceutically acceptable salt thereof and the lopinavir or pharmaceutically acceptable salt thereof, and the second layer comprises the additional anti-HIV agent or pharmaceutically acceptable salt thereof. In some embodiments, the first layer comprises at least 75% by weight of the bilayer tablet, alternatively at least 80% by weight of the bilayer tablet, alternatively at least 85% by weight of the bilayer tablet. In some embodiment, the second layer is 25% or less, alternatively 20% or less, alternatively 15% or less by weight of the bilayer tablet. In some embodiments, the weight of the second layer is 300 mg or less, alternatively 275 mg or less, alternatively 250 mg or less, alternatively 235 mg or less, alternatively 220 mg or less, alternatively 210 mg or less.

For the second layer to be relatively smaller yet contained a desired dose amount of an additional anti-HIV agent, a relatively high percentage or loading of an additional anti-HIV agent can be included in the second layer. A solid pharmaceutical dosage form can be a solid oral dosage form which is administered orally to a patient, having a suitable size, shape and/or coating to facilitate oral administration.

As an aspect of the present invention, a solid pharmaceutical dosage form or a pharmaceutical preparation comprising lopinavir and ritonavir is provided which has a far lower amount of certain excipients than current Kaletra tablets. The solid pharmaceutical dosage form (or pharmaceutical preparation) has less than 1% by weight of a lubricant such as sodium stearyl fumarate, based on the weight of the uncoated tablet. Alternatively or additionally, the solid pharmaceutical dosage form has less than 1.6% by weight of a flow regulator such as colloidal silicon dioxide, based on the weight of the uncoated tablet. Alternatively, the weight percentage of the sodium stearyl fumarate and silicon dioxide is based on the weight of a coated tablet or other dosage form. In some embodiments, a mixture comprising lopinavir, ritonavir, a water soluble polymer, a surfactant, and colloidal silicon dioxide is extruded, and the extrudate is then mixed with an amount of sodium stearyl fumarate that is 1% by weight or less, alternatively 0.8% by weight or less, alternatively 0.7% by weight or less, alternatively 0.6% by weight or less, alternatively 0.4% by weight or less, alternatively 0.2% by weight or less, preferably 0.1% by weight or from 0.05% to 0.15% by weight, relative to total weight of the extrudate, and an amount of colloidal silicon dioxide that is 0.6% by weight or less, alternatively 0.4% by weight or less, alternatively 0.2% by weight or less, alternatively 0.1% by weight or less, alternatively 0.08% by weight or less, preferably from 0.06% to 0.07% by weight, relative to total weight of the extrudate. In other embodiments, the extrudate is then mixed with an amount of sodium stearyl fumarate that is from 0.6% to 0.7% by weight, relative to total weight of the extrudate. Methods of preparing such a solid pharmaceutical dosage form comprising mixing an extrudate comprising lopinavir, ritonavir and other excipients with the foregoing amounts of sodium stearyl fumarate and/or colloidal silicon dioxide are also contemplated.

In some embodiments, a pharmaceutical dosage form comprises two layers of anti-HIV agents. The first layer comprises at least a combination of ritonavir and lopinavir. Lopinavir in the first layer of the pharmaceutical dosage form is present in a suitable dose amount, for example 200, 180, 170 or 160 mg, alternatively 100, 90, 85 or 80 mg in a pediatric dosage form; and ritonavir in the first layer of the pharmaceutical dosage form is present in a suitable dose amount, for example, one-fourth of the amount of lopinavir, such as 50, 45, 42.5 or 40 mg, respectively, alternatively 25, 22.5, 21.25 or 20 mg in a pediatric dosage form, respectively. The second layer of the pharmaceutical dosage form comprises an additional anti-HIV agent. The additional anti-HIV agent in the second layer of the pharmaceutical dosage form is present in an amount effective for its desired therapeutic effect and may be at a relatively high loading as a percentage of the second layer. In some embodiments, the solid pharmaceutical dosage form contains lopinavir in an amount of 200 mg; ritonavir in an amount of 50 mg; and an additional anti-HIV agent in a suitable amount, for example 50 mg, 75 mg or 100 mg. In some embodiments, the solid pharmaceutical dosage form contains lopinavir in an amount of 180 mg; ritonavir in an amount of 45 mg; and an additional anti-HIV agent in a suitable amount, for example 50 mg, 75 mg or 100 mg. In some embodiments, the solid pharmaceutical dosage form contains lopinavir in an amount of 170 mg; ritonavir in an amount of 42.5 mg; and an additional anti-HIV agent in a suitable amount, for example 50 mg, 75 mg or 100 mg. In some embodiments, the solid pharmaceutical dosage form contains lopinavir in an amount of 160 mg; ritonavir in an amount of 40 mg; and an additional anti-HIV agent in a suitable amount, for example 50 mg, 75 mg or 100 mg. In some embodiments, the solid pharmaceutical dosage form contains lopinavir in an amount of 100 mg; ritonavir in an amount of 25 mg; and an additional anti-HIV agent in a suitable amount, for example 25 mg, 37.5 mg or 50 mg. In some embodiments, the solid pharmaceutical dosage form contains lopinavir in an amount of 90 mg; ritonavir in an amount of 22.5; and an additional anti-HIV agent in a suitable amount, for example 25 mg, 37.5 mg or 50 mg. In some embodiments, the solid pharmaceutical dosage form contains lopinavir in an amount of 85 mg; ritonavir in an amount of 21.25; and an additional anti-HIV agent in a suitable amount, for example 25 mg, 37.5 mg or 50 mg. In some embodiments, the solid pharmaceutical dosage form contains lopinavir in an amount of 80 mg; ritonavir in an amount of 20; and an additional anti-HIV agent in a suitable amount, for example 25 mg, 37.5 mg or 50 mg.

In some embodiments, the solid pharmaceutical dosage form consists essentially of ritonavir, lopinavir, and an additional anti-HIV agent (or a pharmaceutically acceptable salt of one or more of the foregoing) and one or more excipients. In some embodiments, the dosage forms do not contain any active ingredient other than ritonavir, lopinavir, and an additional anti-HIV agent (or their salts).

A solid pharmaceutical dosage form can include various excipients. For example, the dosage forms may contain other pharmaceutical additives, such as pharmaceutically acceptable water-soluble polymers, pharmaceutically acceptable surfactants, flow regulators, lubricants, bulking agents (fillers) and disintegrants. When the solid pharmaceutical dosage form is a bilayer tablet, the individual layers may have the same or different excipients, in the same or different weight percentages. For example, in an exemplary bilayer tablet, a layer containing lopinavir and ritonavir may include a pharmaceutically acceptable water-soluble polymer, a pharmaceutically acceptable surfactant, a flow regulator, a bulking agent and a glidant, and a layer containing an additional anti-HIV agent may include a pharmaceutically acceptable water-soluble polymer, a flow regulator, a bulking agent and a glidant, wherein the specific excipients (for example, the flow regulator and glidant) may be the same in both layers, while other excipients (for example, the pharmaceutically acceptable water-soluble polymer and bulking agent) may be different in the different layers.

In some embodiments, a solid pharmaceutical dosage form has a first layer containing lopinavir and ritonavir, and the first layer further comprises from 55% to 75% by weight, alternatively from 68% to 72% by weight, of at least one pharmaceutically acceptable water-soluble polymer, based on total weight of the first layer; from 4% to 10% by weight of at least one pharmaceutically acceptable surfactant; from 0.05% to 1% by weight of at least one lubricant (for example, sodium stearyl fumarate), based on total weight of the first layer; and from 0.01% to 3% by weight of at least one flow regulator (for example, colloidal silicon dioxide), based on total weight of the first layer. In preferred embodiments, the first layer comprises less than 1% by weight of a lubricant (for example, sodium stearyl fumarate), alternatively 0.8% by weight or less, alternatively 0.6% by weight or less, alternatively 0.4% by weight or less, alternatively 0.2% by weight or less, preferably 0.1% by weight, alternatively from 0.1% to 0.2% by weight, relative to total weight of the first layer. Additionally or alternatively, in some embodiments, the first layer comprises less than 1.6% by weight of colloidal silicon dioxide, alternatively 1.5% by weight or less, alternatively 1.2% by weight or less, alternatively 1.1% by weight or less, relative to total weight of the first layer. In other embodiments, the first layer comprises from 1.3% to 1.5%, relative to total weight of the first layer. In other embodiments, the dosage form comprises colloidal silicon dioxide in an amount that is from 0.9% to 1.3% by weight, relative to total weight of the dosage form. In some preferred embodiments of the solid pharmaceutical dosage form, the first layer comprises 55% to 75% by weight, alternatively from 68% to 72% by weight, of N-vinyl pyrrolidone/vinyl acetate copolymer 60:40, based on total weight of the first layer; from 3% to 10% by weight of sorbitan monolaurate, based on total weight of the first layer; from 0.05% to 0.15% by weight of sodium stearyl fumarate, based on total weight of the first layer; and from 1% to 1.2% by weight of colloidal silicon dioxide, based on total weight of the first layer.

In some embodiments, the solid pharmaceutical dosage form has a second layer which comprises from 21.4% to 50% by weight of an additional anti-HIV agent, alternatively from 30% to 40% by weight, preferably 35.7% by weight, based on the weight of the second layer. The second layer can also comprise from 0% to 75% by weight of microcrystalline cellulose, based on total weight of the second layer; from 0% to 50% by weight of lactose, based on total weight of the second layer; from 1.0% to 5.0% by weight of sodium starch glycolate, based on total weight of the second layer; from 0.1% to 2.0% by weight of sodium stearyl fumarate, based on total weight of the second layer; from 0.2% to 3.0% by weight of colloidal silicon dioxide, based on total weight of the second layer; and from 0% to 75% by weight of hydroxypropyl cellulose, based on total weight of the second layer. More preferably, the second layer comprises from 60.1% to 60.3% by weight of microcrystalline cellulose; 3.0% by weight of sodium starch glycolate; 0.8% by weight of sodium stearyl fumarate; and from 0.2% to 0.4% by weight of colloidal silicon dioxide.

In some embodiments, a pharmaceutical preparation or layer containing an additional anti-HIV agent does not contain, or is essentially free of, any pharmaceutically acceptable water-soluble polymer. Alternatively or additionally, a pharmaceutical preparation or layer containing an additional anti-HIV agent does not contain, or is essentially free of, bulking agent. Preferably, the additional anti-HIV agent layer is free of lactose.

In one embodiment, the pharmaceutical dosage form comprises from 5 to 30% by weight of the total dosage form (preferably from 10 to 25% by weight of the total dosage form) of a combination of HIV protease inhibitors, from 50 to 85% by weight of the total dosage form (preferably from 55% to 75% by weight of the total dosage form) of a water-soluble polymer (or any combination of such polymers), from 2 to 20% by weight of the total dosage form (preferably from 3 to 15% by weight of the total dosage form) of the surfactant (or combination of surfactants), and from 0 to 15% by weight of the total dosage form of additives or other excipients.

As yet another aspect of the present invention, a method of preparing a solid pharmaceutical dosage form comprising lopinavir, ritonavir, and an additional anti-HIV agent, or a pharmaceutically acceptable salt of one or more of the foregoing, is provided. The method comprises preparing a homogeneous melt of ritonavir or pharmaceutically acceptable salt thereof, lopinavir or pharmaceutically acceptable salt thereof, a water-soluble polymer(s) and a surfactant(s). The method also comprises extruding the melt to obtain an extrudate, with optional milling of the extrudate. The extrudate is mixed with a flow regulator and a lubricant to form a first pharmaceutical preparation, and the first pharmaceutical preparation is added to a bilayer tablet press. The method also comprises adding a second pharmaceutical preparation to the bilayer tablet press, wherein the second pharmaceutical preparation comprises an additional anti-HIV agent or pharmaceutically acceptable salt thereof. The first pharmaceutical preparation and the second pharmaceutical preparation are compressed together to form a bilayer tablet. In some embodiments, the amount of lubricant (for example, sodium stearyl fumarate) is less than 1% by weight, alternatively less than 0.8% by weight, alternatively 0.7% by weight or less, alternatively 0.6% by weight or less, alternatively 0.4% by weight or less, preferably 0.2% by weight or less, based on the total weight of the dosage form. Alternatively or additionally, in some embodiments of the method, the amount of flow regulator (for example, colloidal silicon dioxide) mixed with the extrudate is 0.6% by weight or less, alternatively 0.4% by weight or less, 0.2% by weight or less, 0.1% by weight or less, 0.09% by weight or less, 0.08% by weight or less, most preferably from 0.06% to 0.07% by weight, based on total weight of the extrudate. The method can comprise the step of initially compressing the first pharmaceutical preparation before compressing the second pharmaceutical preparation, wherein the initial compressing step is at a lower pressure than the compression of the first pharmaceutical preparation and second pharmaceutical preparation together. For example, the initial compression of the first preparation can be at a pressure of 1 to 15 kN, preferably 1 to 2 kN, and the later compression to form the tablet can be at a pressure of 20 to 50 kN, preferably 20 to 35 kN. The compression pressure is partially depending on tablet size and can be adjusted based on tablet size.

In some embodiments, the solid pharmaceutical dosage forms exhibit a release and absorption behavior that may be characterized by high attainable AUC, high attainable Cₘₐₓ (maximum plasma concentration), and low Tₘₐₓ (time to reach maximum plasma concentration). The term “AUC” means “Area Under the Curve” and is used in its normal meaning, as the area under the plasma concentration-time curve from 0 to 24 hours. The term “AUC_{∞}” refers to the area under the plasma concentration time curve (AUC) extrapolated to infinity.

In many embodiments, the solid dosage forms of the invention are characterized by an excellent stability and, in particular, exhibit high resistance against recrystallization or decomposition of lopinavir and ritonavir. In many cases, upon storage for 6 weeks at 40° C. and 75% humidity (e.g., when kept in high density polyethylene (HDPE) bottles without desiccant), the dosage forms do not exhibit any sign of crystallization (as evidenced by DSC or WAXS analysis) and contain at least 98% of the initial content of lopinavir and ritonavir (as evidenced by HPLC analysis).

It should be understood that the above-described embodiments and the following examples are given by way of illustration, not limitation. Various changes and modifications within the scope of the present invention will become apparent to those skilled in the art from the present description.

### Example 1

Table 1 demonstrates the composition of an exemplary first layer of a tablet dosage form of the invention. The extrusion process is similar to that described in Example 2 of U.S. Pat. No. 8,025,899; and the post-extrusion process is similar to that described in Example 3 of U.S. Pat. No. 8,025,899, and the final powdery blend is pre-compressed into the first layer. The entire content of U.S. Pat. No. 8,025,899 is incorporated herein by reference.

TABLE 1
First Layer

| | | | |
|---|---|---|---|
| | Unit Formula | Wt % based on | Wt % based on |
| Material | (Per Tablet) | extrudate | uncoated tablet |

Extrusion

| | | | | |
|---|---|---|---|---|
| Active Agents & | | | | |
| Excipients | | | | |
| Lopinavir^{a} | 200.0 | mg | 16.67% | 16.39% |
| Ritonavir^{a} | 50.0 | mg | 4.17% | 4.10% |
| Excipients | | | | |
| Copovidone, K | 853.8 | mg | 71.16% | 69.98% |
| value 28 | | | | |
| Sorbitan | 83.9 | mg | 7.0% | 6.88% |
| monolaurate | | | | |
| Colloidal silicon | 12.0 | mg | 1.00% | 0.98% |
| dioxide | | | | |
| Total Extrudate | 1199.7 | mg | 100% | |
| Weight | | | | |

Post Extrusion

| | | | | |
|---|---|---|---|---|
| Sodium stearyl | 1.23 | mg | 0.1025% | 0.1008% |
| fumarate | | | | |
| Colloidal silicon | 0.80 | mg | 0.0667% | 0.0656% |
| dioxide | | | | |
| Uncoated Tablet | 1201.7 | mg | | 100% |
| Weight | | | | |

^{a}Factored for chemical potency

Table 2 demonstrates the composition of an exemplary second layer of a tablet dosage form of the invention. The materials in Table 2 can be blended and/or milled, and then compressed against the first layer (Table 1) to form a bilayer tablet core.

TABLE 2
Second Layer

| Material | Wt % |
|---|---|
| Lamivudine | 21.4-50 |
| Microcrystalline cellulose | 0-75 |
| Lactose | 0-50 |
| Sodium starch glycolate | 1.0-5.0 |
| Sodium stearyl fumarate | 0.1-2.0 |
| Colloidal silicon dioxide | 0.2-1.0 |
| Hydroxypropyl cellulose | 0-20 |

Table 3 represents the compositions of two other exemplary second layers. Each composition can be blended and/or milled, and then compressed against the first layer (Table 1) to form a bilayer tablet core.

TABLE 3
Second Layer

| Material | Wt % | Wt % |
|---|---|---|
| Lamivudine | 35.7 | 35.7 |
| Microcrystalline cellulose | 60.1 | 60.3 |
| Lactose | 0 | 0 |
| Sodium starch glycolate | 3.00 | 3.00 |
| Sodium stearyl fumarate | 0.80 | 0.80 |
| Colloidal silicon dioxide | 0.40 | 0.20 |
| Hydroxypropyl cellulose | 0.00 | 0.00 |

A film coat can be added to the bilayer tablet core. The amount can be any suitable amount, for example, 20 mg to 28 mg (including 22 mg, 23 mg, 24 mg, 25 mg or 26 mg), per tablet.

### Example 2

Copovidone (N-vinyl pyrrolidone/vinyl acetate copolymer 60:40) (853.8 parts by weight) can be blended with Span 20 (Sorbitan monolaurate) (83.9 parts by weight) in a Diosna high-shear mixer. The resulting granules are mixed with ritonavir (50 parts by weight), lopinavir (200 parts by weight) and colloidal silica (12 parts by weight). The powdery mixture is then fed into a twin-screw extruder (screw diameter 18 mm) at a rate of 2.1 kg/h and a melt temperature of 119° C. The extrudate is cut into pieces and allowed to solidify. The extruded pieces are milled to a mean particle size of about 200 μm, using a high impact hammer mill (more particularly, a Fitzmill). The milled material can be blended in a bin blender with sodium stearyl fumarate (1.23 parts by weight) and colloidal silica (0.8 parts per weight) for 5 min at 6 rpm to provide a pharmaceutical preparation suitable for pre-compression to make the first layer. More particularly, a portion of the milled extrudate (for example, about 10% of the milled extrudate) can be first combined with the sodium stearyl fumarate and colloidal silica and milled using a Quadro Comil U10 (Screen Type/ID: Round Hole/7A094R03741; Impeller Type/ID: Round Bar/7A160110001; Speed (Target): 1400 RPM). Alternatively the mixture can be milled using a Quadro CoMill U20 with a 2.4 mm screen and an impeller. The remaining portion of the milled extrudate is then added and blended to provide a powdery pharmaceutical preparation suitable for pre-compression to make the first layer.

### Example 3

Lamivudine (3TC) (35.7 parts by weight) was combined with sodium starch glycolate (3 parts by weight), sodium stearyl fumarate (0.80 parts by weight) and colloidal silica (0.40 parts by weight) and manually mixed. The resulting mixture was milled using a Quadro Comil U10 (Screen Type/ID: Round Hole/7A094R03741; Impeller Type/ID: Round Bar/7A160110001; Speed (Target): 1400 RPM). The mill screen was then rinsed with 5 parts by weight of Emcocel 90M (microcrystalline cellulose having a normal particle size of 100, a moisture content of 3.0 to 5.0%, and a loose bulk density of 0.28 to 0.33 g/cc). The resulting mixture was blended with Emcocel 90M (55.10 parts by weight) for 20 minutes at 15 RPM to provide a pharmaceutical preparation suitable for compressing into the second layer of a tablet dosage form of the invention.

### Example 4

A pharmaceutical preparation according to Example 2 and a pharmaceutical preparation according to Example 3 were placed in a power-assisted automatic bilayer tablet press to form a bilayer tablet comprising 1202 mg of the first layer and 210 mg of the second layer. The pharmaceutical preparation according to Example 2 (85.13 parts by weight), containing a lopinavir/ritonavir blend, was fed to a Riva Piccola bilayer rotary press and partially compressed. Then the pharmaceutical preparation according to Example 3 (14.87 parts by weight), containing a lamivudine blend, was fed to the bilayer rotary press, and the two blends were together subjected to a hard compression which yielded a bilayer tablet core. The bilayer tablet core was then film-coated in a coating pan by spraying an aqueous dispersion for film coating (Opadry®, available from Colorcon) at an inlet temperature of 75° C.

The release profiles of lopinavir, ritonavir, and lamivudine from a tablet thus prepared were tested. The release rate of each of these active ingredients was essentially the same as that from a combination of commercially available tablets—namely a Kaletra tablet (containing 200 mg lopinavir and 50 mg ritonavir per tablet) plus one half of an Epivir tablet (containing 150 mg lamivudine per tablet). Drug dissolutions were measured using the USP type 2 dissolution apparatus, at 75 rpm in 900 ml of 0.06M POE10LE for 120 minutes at 37° C.

### Example 5

Copovidone (N-vinyl pyrrolidone/vinyl acetate copolymer 60:40) (853.8 parts by weight) can be blended with Span 20 (Sorbitan monolaurate) (83.9 parts by weight) in a Diosna high-shear mixer. The resulting granules are mixed with ritonavir (50 parts by weight), lopinavir (200 parts by weight) and colloidal silica (12 parts by weight). The powdery mixture is then fed into a twin-screw extruder (screw diameter 18 mm) at a rate of 2.1 kg/h and a melt temperature of 119° C. The extrudate can be cut into pieces and allowed to solidify. The extruded pieces are milled to a mean particle size of about 200 μm, using a high impact hammer mill. The milled material can be blended in a bin blender with sodium stearyl fumarate (9.3 parts by weight) and colloidal silica (5.1 parts per weight) for 5 min at 6 RPM to provide a pharmaceutical preparation suitable for pre-compression to make the first layer of a tablet dosage form of the invention. More particularly, a portion of the milled extrudate (for example, about 10% of the milled extrudate) can be first combined with the sodium stearyl fumarate and colloidal silica and milled using a Quadro Comil U10 (Screen Type/ID: Round Hole/7A094R03741; Impeller Type/ID: Round Bar/7A160110001; Speed (Target): 1400 RPM). Alternatively the mixture can be milled using a Quadro CoMill U20 with a 2.4 mm screen and an impeller. The remaining portion of the milled extrudate is then added and milled to provide a powdery pharmaceutical preparation suitable for pre-compression to make the first layer.

### Example 6

Lamivudine (3TC) (35.7 parts by weight) was combined with sodium starch glycolate (3 parts by weight), sodium stearyl fumarate (0.80 parts by weight) and colloidal silica (0.20 parts by weight) and manually mixed. The resulting mixture was milled using a Quadro Comil U10. The mill screen was then rinsed with 5 parts by weight of Emcocel 90M (microcrystalline cellulose having a normal particle size of 100, a moisture content of 3.0 to 5.0%, and a loose bulk density of 0.28 to 0.33 g/cc). The resulting mixture was blended with Emcocel 90M (55.30 parts by weight) for 20 minutes at 15 RPM to provide a pharmaceutical preparation suitable for compressing into the second layer of a tablet dosage form of the invention.

### Example 7

A pharmaceutical preparation according to Example 5 and a pharmaceutical preparation according to Example 6 were placed in a power-assisted automatic bilayer tablet press to form a bilayer tablet. The pharmaceutical preparation according to Example 5 (85.25 parts by weight), containing a lopinavir/ritonavir blend, was fed to a Riva Piccola bilayer rotary press. The pharmaceutical preparation according to Example 6 (14.75 parts by weight), containing the lamivudine blend, was fed to the bilayer rotary press. The pharmaceutical preparation according to Example 5 was subjected to an initial compression, and then the two pharmaceutical preparations were together subjected to a harder compression to yield a bilayer tablet core. The bilayer tablet core was then film-coated in a coating pan by spraying an aqueous dispersion for film coating (Opadry®, available from Colorcon) at an inlet temperature of 75° C.

The release profiles of lopinavir, ritonavir, and lamivudine from a tablet thus prepared were tested. The release rate of each of these active ingredients was essentially the same as that from a combination of commercially available tablets—namely a Kaletra tablet (containing 200 mg lopinavir and 50 mg ritonavir per tablet) plus one half of an Epivir tablet (containing 150 mg lamivudine per tablet). Drug dissolutions were measured using the USP type 2 dissolution apparatus, at 75 rpm in 900 ml of 0.06M POE10LE for 120 minutes at 37° C.

As compared to the commercial Kaletra tablet (which contains about 12.3 parts by weight of lubricant, corresponding to about 1% by weight of the total tablet), the bilayer tablet of Example 7 (which contains 9.3 parts by weight of lubricant, corresponding to about 0.76% by weight of the lopinavir/ritonavir layer) required lower compression force and showed more desired flower properties. Example 4 (which contains only 1.23 parts by weight of lubricant, corresponding to about 0.1% by weight of the lopinavir/ritonavir layer) required even lower compression force and showed more desired flower properties than Example 7.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A tablet dosage form comprising a first layer and a second layer, wherein the first layer comprises (1) from 160 to 200 mg lopinavir, preferably 170 or 180 mg lopinavir, (2) ritonavir, (3) a pharmaceutically acceptable hydrophilic polymer, (4) a pharmaceutically acceptable surfactant and (5) no more than 0.9% by weight of one or more lubricants, and the second layer comprises another therapeutic agent.

2. The tablet dosage form of claim 1, wherein the first layer comprises no more than 0.5% by weight of one or more lubricants.

3. The tablet dosage form of claim 1, wherein the first layer comprises no more than 0.2% by weight of one or more lubricants.

4. The tablet dosage form of claim 1, wherein the first layer comprises no more than 0.1% by weight of one or more lubricants.

5. The tablet dosage form of claim 3, wherein the weight ratio of the second layer to the first layer is no more than 1:4.

6. The tablet dosage form of claim 3, wherein the weight ratio of the second layer to the first layer is no more than 1:5.

7. The tablet dosage form of claim 3, wherein the dosage form is no more than 1.7 g, and the first layer is at least 1 g.

8. The tablet dosage form of claim 3, wherein the dosage form is no more than 1.6 g, and the first layer is at least 1 g.

9. The tablet dosage form of claim 3, wherein the dosage form is no more than 1.5 g, and the first layer is at least 1 g.

10. The tablet dosage form of claim 3, wherein the dosage form is no more than 1.5 g, and the first layer is at least 1.1 g.

11. The tablet dosage form of claim 10, wherein the weight ratio of the second layer to the first layer is no more than 1:4.

12. The tablet dosage form of claim 10, wherein the weight ratio of the second layer to the first layer is no more than 1:5.

13. The tablet dosage form of claim 3, wherein the first layer includes a solid dispersion comprising ritonavir, lopinavir, said hydrophilic polymer and said surfactant.

14. The tablet dosage form of claim 3, wherein the first layer includes a solid solution comprising ritonavir, lopinavir, said hydrophilic polymer and said surfactant.

15. The tablet dosage form of claim 3, wherein the first layer includes a glassy solution comprising ritonavir, lopinavir, said hydrophilic polymer and said surfactant.

16. The tablet dosage form of claim 15, wherein said hydrophilic polymer is copovidone, and said surfactant is sorbitan monolaurate.

17. The tablet dosage form of claim 16, wherein said one or more lubricants are sodium stearyl fumarate.

18. The tablet dosage form of claim 17, wherein the first layer comprises 50 mg ritonavir.

19. A pediatric tablet dosage form comprising a first layer and a second layer, wherein the first layer comprises (1) from 80 to 100 mg lopinavir, preferably 85 or 90 mg lopinavir, (2) ritonavir, (3) a pharmaceutically acceptable hydrophilic polymer, (4) a pharmaceutically acceptable surfactant and (5) no more than 0.9% by weight of one or more lubricants, and the second layer comprises another therapeutic agent.

20. A process of making a tablet dosage form, comprising compressing a first layer and a second layer under a compression force of no more that 30 kN, wherein the first layer comprises (1) from 160 to 200 mg lopinavir, preferably 170 or 180 mg lopinavir, (2) ritonavir, (3) a pharmaceutically acceptable hydrophilic polymer, (4) a pharmaceutically acceptable surfactant and (5) no more than 0.9% by weight of one or more lubricants, and the second layer comprises another therapeutic agent.

21. A method of treating HIV infection, comprising administering a tablet dosage form of claim 1 to a HIV patient.
